(19) United States
(12) Patent Application Publication
(10) Pub. No.: US 2019/0053723 A1
(43) Pub. Date: Feb. 21, 2019

(54) Clamping Device, System and Method for Controlling Venous Blood Flow, Improving Venous Dilation and Effecting Blood Pressure Measurement

(71) Applicant: Tournicare Pty Ltd, Camberwell, Victoria (AU)

(72) Inventors: Niels van Sparrentak, Balwyn, Victoria (AU); Rohan White, Hawthorn, Victoria (AU)

(21) Appl. No.: 15/764,267

(22) PCT Filed: Sep. 29, 2016
(86) PCT No.: PCT/AU2016/050911

§ 371 Date: Mar. 28, 2018

(30) Foreign Application Priority Data
Oct. 1, 2015 (AU) 2015904004

(51) Int. Cl. A61B 5/022; A61B 5/107; A61B 5/00

(52) U.S. Cl. CPC A61B 5/02233; A61B 5/02208; A61B 5/02007; A61B 5/6824; A61B 5/6828; A61B 5/107

(57) ABSTRACT

A clamping device for applying pressure to a human limb. The device comprises a first rigid part having a first inner profile and a second rigid part generally facing the first inner profile. The first and second inner profiles are arranged to apply clamping pressure against the human limb when the device is in the clamped position and thereby apply clamping pressure to blood vessels in the limb. The device further comprises a coupling portion that couples the first and second rigid parts together while allowing relative movement of the first and second parts between a clamped position and an unclamped position. An expandable element is arranged at least partly along at least one of the first inner profile and the second inner profile. The expandable element is inflatable to apply additional pressure to the limb, and deflatable to reduce the additional pressure, when the clamping device is positioned on the limb. The device further comprises at least one sensing component.

### TECHNICAL FIELD

Described embodiments generally relate to clamping devices, systems and methods for controlling venous blood flow and methods of using such clamping devices. In particular, embodiments relate to clamping devices that are also configured for blood pressure measurements.

### BACKGROUND

Tourniquets have been used traditionally for many years when performing venepuncture. Such tourniquets generally consist of a flexible band to encircle the arm or lower limb and compress the arm or lower limb around the circumference of that limb. Tourniquets can be used to increase the vein diameter to improve visibility of the vein and provide a larger vein to thereby aid venepuncture. Tying the tourniquet, however, may be a cumbersome and time consuming process.

The blood pressure of a subject is often measured before venepuncture. This can be performed using a conventional inflatable cuff that is secured around a limb and blood pressure can be measured using a sphygmomanometer that makes use of the oscillometric method or the auscultatory method. However, the inflatable cuff is manually secured around a limb and the tightness with which it is secured, as well as the cuff size, can affect the accuracy of blood pressure measurements.

The Applicant's co-owned International application, published as WO 2014/179830, discloses a clamping device that can be used in place of a traditional tourniquet or inflatable cuff. The contents of WO 2014/179830 are incorporated herein in their entirety.

Any discussion of documents, acts, materials, devices, articles or the like which has been included in the present specification is not to be taken as an admission that any or all of these matters: form part of the prior art base; were common general knowledge in the field relevant to the present disclosure as it existed before the priority date of each claim of this application; or could have been understood, regarded as relevant or reasonably expected to have been combined by a person skilled in the art.

### SUMMARY

Some embodiments relate to a clamping device for applying pressure to the human limb comprising: a first rigid part having a first inner profile, a second rigid part generally facing the first inner profile, wherein the first and second inner profiles are arranged to apply clamping pressure against the human limb when the device is in the clamped position and thereby apply clamping pressure to blood vessels in the limb; a coupling portion that couples the first and second rigid parts together while allowing relative movement of the first and second parts between a clamped position and an unclamped position; an expandable element arranged at least partly along at least one of the first inner profile and the second inner profile; wherein the expandable element is inflatable to apply additional pressure to the limb, and deflatable to reduce the additional pressure, when the clamping device is positioned on the limb; and at least one sensing component.

In some embodiments, the at least one sensing component includes a spatial sensing component adapted to sense or indicate a spatial dimension value for either a diameter or circumference of the human limb. In some embodiments, the clamping device includes a meter for indicating the spatial dimension value. In some embodiments, the meter includes a graduated scale on either the first rigid part or the second rigid part and the spatial sensing component is connected to an elongate pointer arranged to indicate the spatial dimension value on the graduated scale. In some embodiments, the spatial sensing component is a spatial sensor adapted to provide a first output indicative of a spatial dimension value for the spatial dimension.

In some embodiments, the clamping device further comprises a pressure sensing component comprising a pressure sensor for sensing pressure. In some embodiments, the pressure sensor is adapted to provide a second output indicative of pressure. In some embodiments, the second output comprises an output indicative of the clamping pressure applied by the clamping device to the human limb.

In some embodiments, the clamping device further comprises: a processor adapted to receive the second output; and an indicator, wherein the processor is configured to trigger the indicator to indicate when a predetermined clamping pressure is reached. In some embodiments, the indicator provides at least one of a visible or audible output.

In some embodiments, the clamping device is adapted to provide at least one of the first output or second output to a computing device. In some embodiments, the spatial sensor and the pressure sensor are adapted to be electrically coupled to the computing device to thereby provide the at least one of the first and second output. In some embodiments, the clamping device further comprises at least one transmitter for providing the at least one of the first and second output to the computing device.

In some embodiments, the clamping device further comprises a display coupled to the at least one sensing component to receive at least one of the first output and second output to thereby display at least one of the spatial dimension value and the clamping pressure value. In some embodiments, the display includes either an electro-mechanical meter or an electronic display.

In some embodiments, the expandable element is adapted to be connected to a sphygmomanometer for inflating and deflating the expandable element and to determine a set of blood pressure values.

In some embodiments, the clamping device further comprises: a pump in fluid connection with the expandable element for inflating and deflating the expandable element; and a controller connected to the pump for controlling the pump to inflate and deflate the expandable element in a predetermined manner for blood pressure measurements.

In some embodiments, the clamping device further comprises: a processor electrically coupled to the pressure sensor, wherein the pressure sensor is pneumatically coupled to the expandable element, and wherein the controller and the processor are adapted to determine a set of corrected blood pressure values using the second output from the pressure sensor and the spatial dimension value.

In some embodiments, the clamping device further comprises: a pump in fluid connection with the expandable element for inflating and deflating the expandable element; a controller connected to the pump for controlling the pump to inflate and deflate the expandable element in a predetermined manner for blood pressure measurements; a further pressure sensor pneumatically coupled to the expandable element; and a processor electrically coupled to the further pressure sensor, wherein the controller and the processor are adapted to determine a set of corrected blood pressure values using an output from the further pressure sensor and the spatial dimension value.

In some embodiments, the spatial sensing component is a spatial sensor coupled to the processor and adapted to provide a first output indicative of a spatial dimension value of the spatial dimension to the processor. In some embodiments, the display is adapted to display the set of corrected blood pressure values.

Some embodiments relate to a blood pressure monitoring system for calculating a corrected blood pressure value in a human limb, the system comprising: the clamping device according to any one of the preceding claims, and a computing device for calculating the corrected blood pressure value, wherein the computing device includes: an input component configured to receive a first value indicative of a cross-sectional dimension of the human limb, a second value indicative of a clamping pressure applied by the clamping device, and a set of blood pressure values, a first processor; and a storage medium including instructions for the processor to calculate a set of corrected blood pressure values using the first value, second value and set of blood pressure values.

In some embodiments, the computing device further comprises a display for displaying the set of corrected blood pressure values.

Some embodiments relate to a method of improved venous dilation comprising: applying a clamping device or an automated inflatable cuff around an upper section of an arm of a human subject at a clamping pressure to create some level of hypoxia in the arm, wherein the clamping pressure is applied at a first pressure substantially equal to the systolic arterial pressure of the arteries in the upper section of the arm; maintaining the level of hypoxia in the arm for a first period of time greater than 20 seconds; releasing the clamping pressure applied by the clamping device or an automated inflatable cuff for a second period of time greater than 5 seconds; subsequent to the second period, increasing the clamping pressure to a second pressure less than the first pressure to thereby improve venous dilation.

In some embodiments of the method of improved venous dilation, the clamping device is the clamping device herein described.

In some embodiments of the method of improved venous dilation, the first period of time is less than 60 seconds. In some embodiments of the method of improved venous dilation, the second period is less than 60 seconds. In some embodiments, the second period is in the range of 10-20 seconds.

In some embodiments, releasing the first clamping pressure comprises releasing the clamping pressure in less than 5 seconds. In some embodiments, releasing the first clamping pressure comprises reducing the first clamping pressure by at least 80%. In some embodiments, the second pressure is between 60 and 140 mmHg. In some embodiments, the method further comprises determining the systolic value in the upper section of the arm and the second pressure is between 60% and 90% of the determined systolic pressure value.

Some embodiments relate to a method of controlling the rate of venous blood outflow comprising: applying a clamping device as herein described or an automated inflatable cuff at a location on an upper section of an arm of a human subject; puncturing a vein in the arm with a needle at a point distal from the location on the arm that the clamping device is applied; with the needle in position, inflating an expandable element on the clamping device or the automated inflatable cuff to apply an applied pressure that is below an arterial pressure in the upper arm; collecting blood with a collection system; and adjusting the applied pressure to control the rate of venous blood outflow.

In some embodiments, the method of controlling the rate of venous blood outflow comprises applying a pressure of approximately 50 mmHg.

### BRIEF DESCRIPTION OF THE DRAWINGS

Embodiments are described in further detail below, by way of example, with reference to the accompanying drawings, in which:

FIG. 1A is a rough anatomical cross-sectional view across an upper arm in an uncompressed state;

FIG. 1B is a rough anatomical cross-sectional view similar to FIG. 1A, showing the upper arm in a compressed state.

FIG. 2A is a perspective view of a clamping device according to some embodiments;

FIG. 2B is a cross-sectional view of a liner part of the device of FIG. 2A;

FIG. 3A is a perspective view of the device of FIG. 2A, shown from a different angle;

FIG. 3B is a perspective view of the device of FIG. 2A, shown from a perspective view similar to that of FIG. 3A, but with a cushioning liner absent;

FIG. 4A is a further perspective view of the device of FIG. 2A, shown in an open or unclamped position;

FIG. 4B is a view of the device similar to that shown in FIG. 4A but shown in a compressed or clamped position;

FIG. 5A is a schematic view illustrating placement of the device of FIG. 2A about an uncompressed upper arm, with the device in the unclamped position;

FIG. 5B is a schematic illustration of the clamping device similar to FIG. 5A but shown in a clamped position, in which the upper arm is compressed;

FIG. 6 is a perspective view of a clamping device according to some alternative embodiments;

FIG. 7 is a partial cut away perspective view of the device of FIG. 2A illustrating biasing components and retention components;

FIG. 8 is a cross-sectional view of part of the device of FIG. 2A further illustrating the biasing and retention components;

FIG. 9 is a perspective view of a clamping device according to further alternative embodiments;

FIG. 10 is a schematic illustration of a clamping device according to still further embodiments;

FIGS. 11A, 11B and 11C are schematic illustrations of a clamping device according to still further embodiments, showing open, partially clamped and fully clamped positions;

FIG. 12 is a schematic illustration of a kit comprising a cradle and a clamping device according to some embodiments mounted or positioned on the cradle;

FIG. 13A is a perspective schematic illustration of a clamping device according to some embodiments;

FIG. 13B is a perspective schematic illustration of a kit comprising a disposable clamp cover according to some embodiments and the clamping device of FIG. 13A;

FIG. 14 is a schematic illustration of a container housing disposable covers for use with the kit of FIG. 13B;

FIG. 15 is a schematic illustration of a kit comprising a disposable clamp cover according to some embodiments and the clamping device of FIG. 2A;

FIG. 16A is a schematic view illustrating a clamping device according to further embodiments, showing placement of the device about an uncompressed upper arm, with the device in the unclamped position;

FIG. 16B is a schematic illustration of the clamping device similar to FIG. 16A but shown in a clamped position, in which the upper arm is compressed;

FIG. 17A is a first perspective view of the clamping device of FIG. 16A, shown in an open position;

FIG. 17B is a second perspective view of the clamping device of FIG. 16A, shown in an open position;

FIG. 18 is a close up perspective view of a retention mechanism of the clamping device of FIG. 16A;

FIG. 19 is a side view of the clamping device of FIG. 16A, shown in a clamped position and illustrating a release position of the retention mechanism to allow the device to adopt the unclamped position;

FIG. 20 is a side view of the clamping device of FIG. 16A, shown in a clamped position and illustrating a breakaway release of the retention mechanism to allow the deice to adopt the unclamped position;

FIG. 21 is a schematic view similar to FIG. 5A but illustrating a further example clamping device that includes a pressure sensor;

FIG. 22 is an example schematic circuit diagram of the pressure sensor shown in FIG. 21;

FIG. 23A is a perspective view of a further example clamping device;

FIG. 23B is a perspective view of the clamping device of FIG. 23A but shown in an inverted position;

FIG. 24 is a schematic illustration of an example expandable element usable in example clamping devices;

FIG. 25 is a schematic illustration of the expandable element of FIG. 24, illustrating the expandable element in an expanded state;

FIG. 26 is a perspective view of a clamping device according to further embodiments;

FIG. 27 is a perspective partial cut-away view of the clamping device of FIG. 26;

FIG. 28 is a further perspective view of the clamping device of FIG. 26;

FIG. 29 is a cross-sectional view of the clamping device of FIG. 28;

FIG. 30 is a schematic block diagram of components of the clamping device of FIG. 26;

FIG. 31 is a schematic block diagram of components of a clamping device similar to FIG. 26 but including voice control features; and

FIG. 32 is a schematic block diagram of components of a clamping device similar to FIG. 26 but including features to allow control of the clamping device by a handheld electronic device;

FIG. 33 is a schematic block diagram of a blood pressure monitoring system including a clamping device according to some embodiments;

FIG. 34 is a schematic block diagram of a blood pressure monitoring system including a clamping device according to some embodiments;

FIG. 35 is a schematic block diagram of a blood pressure monitoring system including a clamping device according to some embodiments;

FIG. 36 is a flow diagram of a method for controlling venous dilation according to some embodiments;

FIG. 37 is a flow diagram of a method for controlling venous blood flow according to some embodiments.

### DETAILED DESCRIPTION

Described embodiments generally relate to clamping devices, systems and methods for controlling venous blood flow and methods of using such clamping devices. In particular, embodiments relate to clamping devices that are also configured for blood pressure measurements.

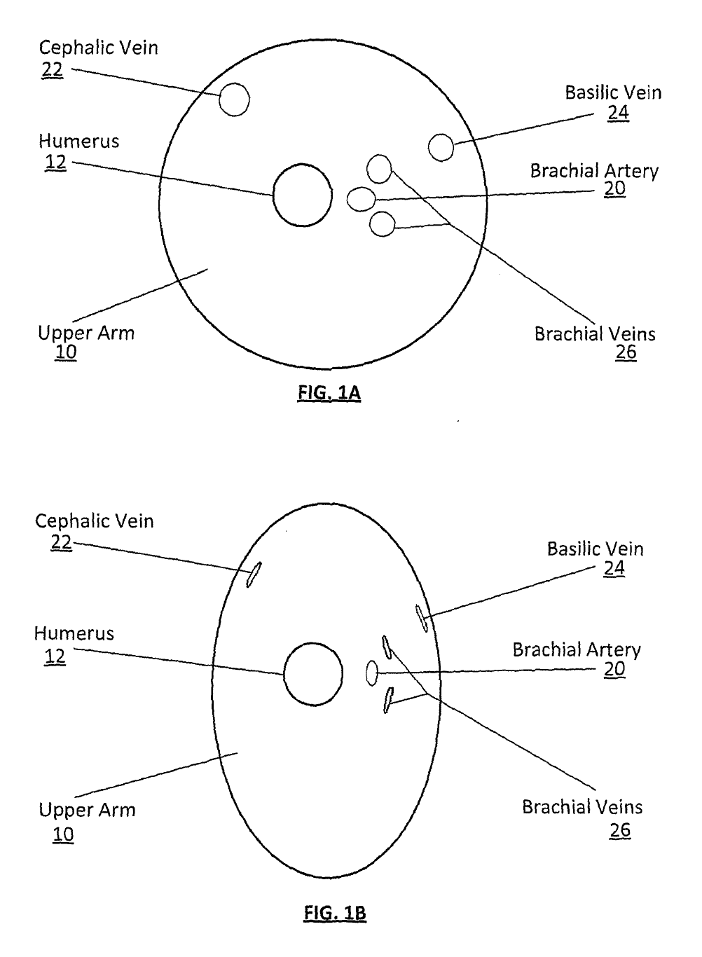

Referring firstly to FIGS. 1A and 1B, certain parts of the anatomy of the upper human arm are discussed for purposes of illustration of an intended use of the clamping device according to some embodiments.

A human upper arm 10 generally has a centrally positioned humerus bone 12 around which tissues are arranged including muscles, veins and arteries. A significant artery in the upper arm 10 is the brachial artery 20 which is generally located deeper within the flesh of the upper arm 10 than the veins, such as the cephalic vein 22, basilica vein 24 and brachial veins 26. FIG. 1A shows the upper arm in an uncompressed state 4a and FIG. 1B shows the upper arm 10 in a laterally and medially compressed state 4b.

As is roughly illustrated in FIG. 1B, in the compressed state 4b, the veins closer to the surface of the upper arm 10 tend to become more compressed when compression is applied to the external lateral and medial surfaces of the upper arm 10. This compression tends to reduce blood flow in the cephalic vein 22, basilic vein 24 and brachial veins 26, which tends to have the effect of reducing blood flow in those veins back to the heart. Since there is greater fluid pressure in the brachial artery 20 and it is positioned more deeply in the arm, there tends to be less choking of the blood flow through the brachial artery 20 as a result of inwardly applied compression to the exterior of the arm, when compared to venous blood flow under such compression. Generally, the cephalic vein is positioned on an upper lateral (outer) side of the arm, while the brachial and basilic veins are positioned on the medial (inner) side of the arm.

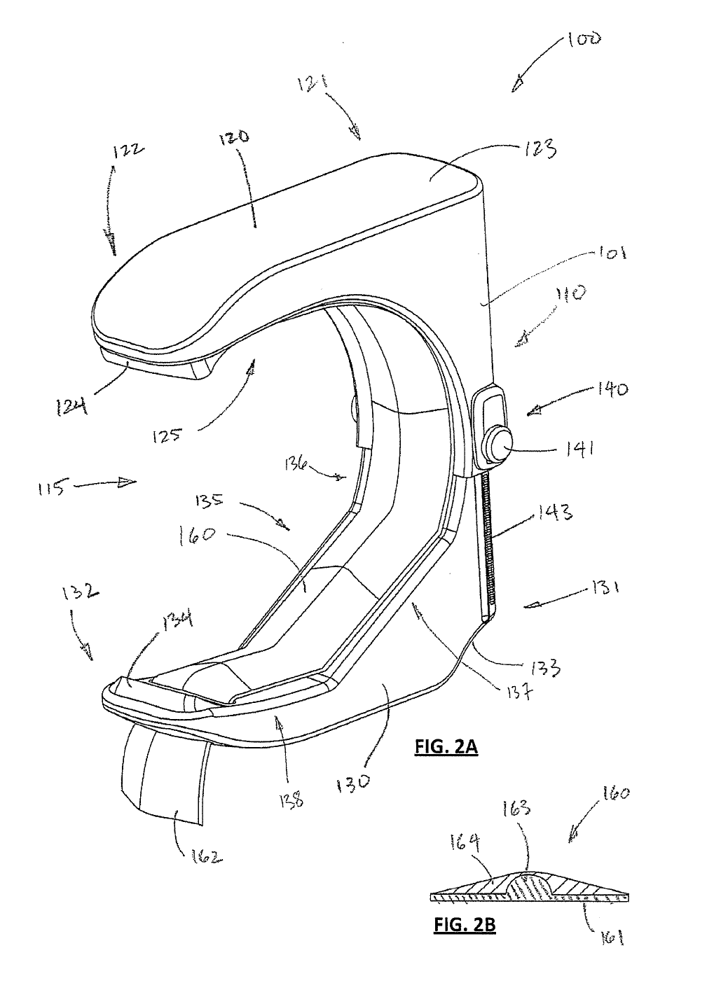

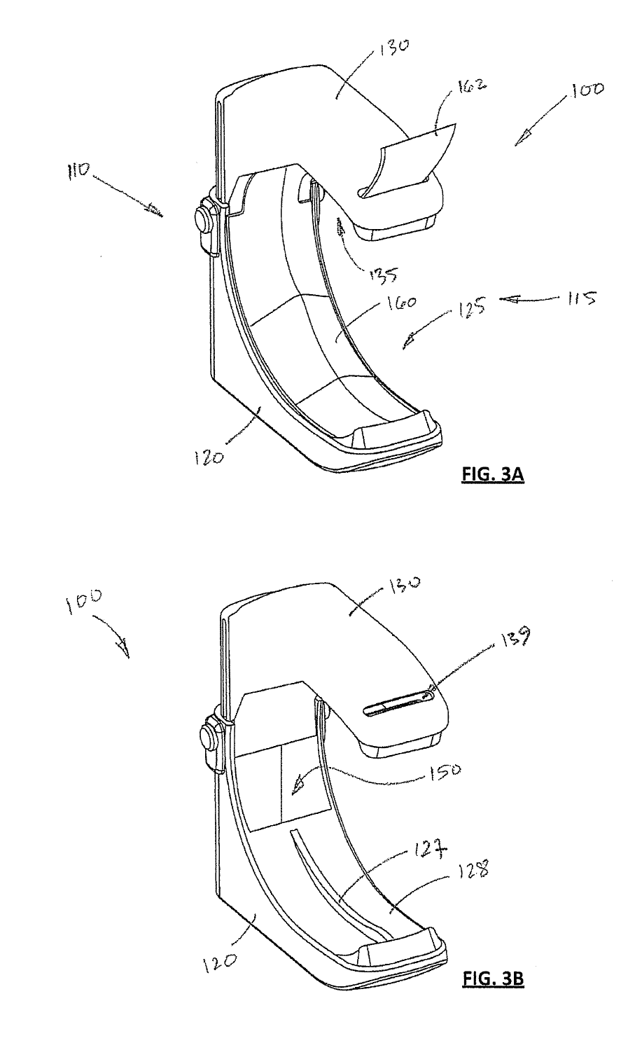

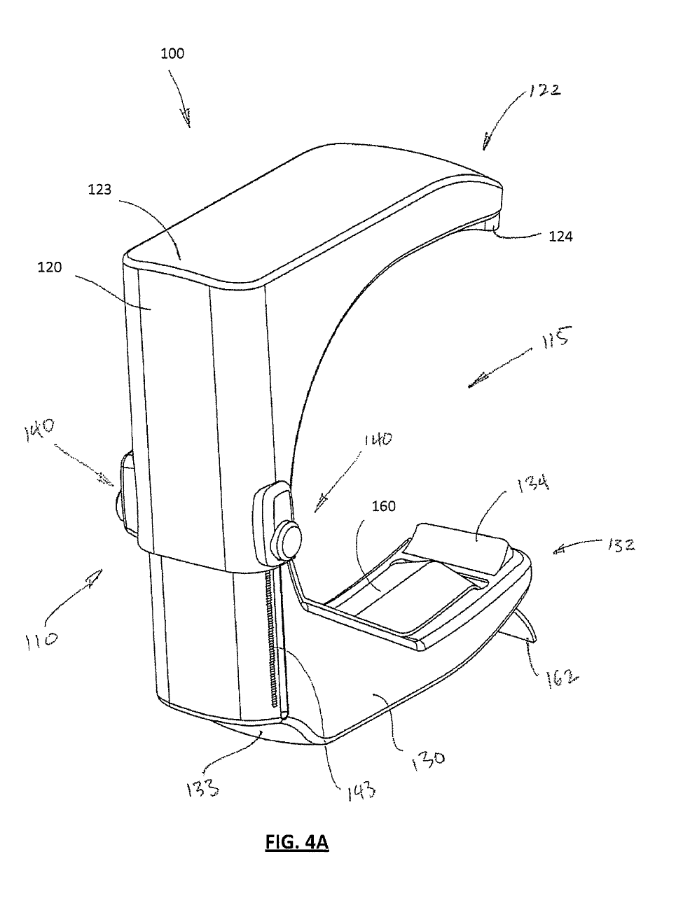

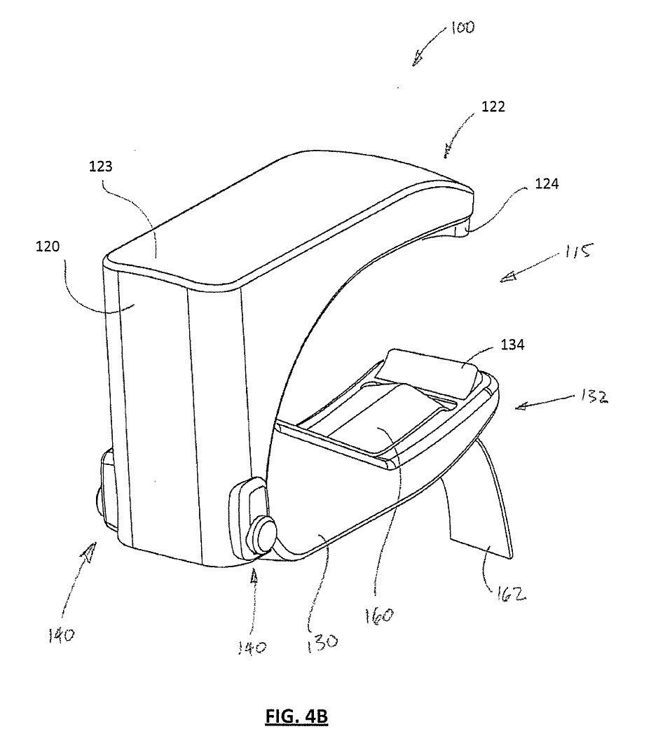

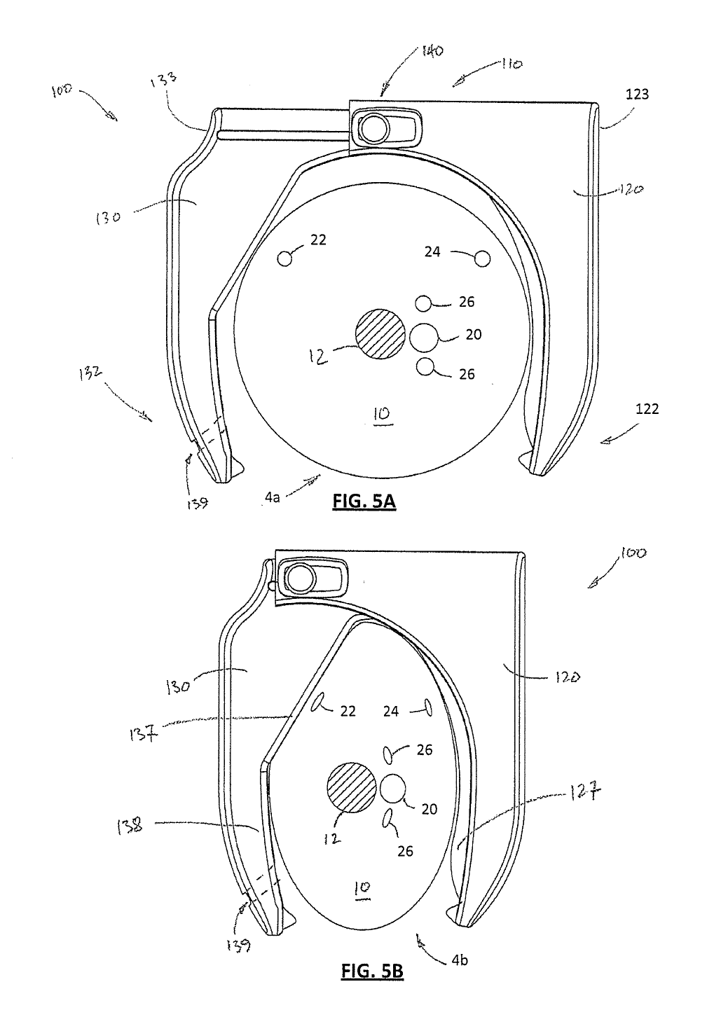

With reference to FIGS. 2A, 2B, 3A, 3B, 4A, 4B, 5A and 5B, a clamping device 100 according to some embodiments is shown and described in further detail. The clamping device 100 generally comprises a first rigid part 120 and a second rigid part 130 that are coupled by coupling or bridge portion 110. The first and second parts 120, 130 may be described as arms or jaws because of their opposed relation and their function of clamping about a limb. As is shown in the Figures, the device 100 is generally approximately U-shaped, with the coupling or bridge portion 110 joining the first and second jaws 120, 130 at an apex of the U-shape.

Device 100 (or another device embodiment described herein) is generally configured to be movable between an unclamped position, in which the jaws 120, 130 are spaced widely enough to allow the device 100 to be placed about or partially about a human limb, such as an upper arm, and a clamped position in which the jaws 120, 130 press toward and against the lateral and medial surfaces of the limb. While embodiments are generally described as being configured for clamping a human upper arm, embodiments may also be configured for clamping other limb parts such as a forearm, a lower leg or an upper leg, for example.

The device 100 is generally formed to have a rigid body 101 that is formed of two main movable parts, comprising the first and second jaws 120, 130. The coupling or bridge portion 110 comprises first and second coupling parts that are each coupled, connected to or integrally formed with the jaws 120, 130, such that relative movement of the first and second coupling parts when the device 100 transitions between the clamped and unclamped positions, corresponds with relative movement between the first and second jaws 120, 130.

The first jaw 120 has a base portion 121 that forms part of the coupling or bridge portion 110. The second jaw 130 also has a base portion 131 that forms part of the bridge or coupling portion 110. The first jaw 120 also has a distal portion 122 at a free end distal of the base portion 121. The first jaw 120 defines a generally non-linear inner profile 125 that faces an opposite non-linear inner profile 135 defined by the opposing second jaw 130.

On an outward face of the base portion 120, there is a land 123 and on an outward face of the base portion 131 of the second jaw 130, there is another land 133. The lands 123, 133 are generally arranged to be manually compressible by a human hand, such that a thumb can be placed on one of the lands 123, 133, while one or more fingers are placed on the opposite land 123, 133 so that manual force can be used to squeeze and move the jaws 120, 130 toward each other and thereby move the device 100 into a clamped position. In some embodiments, the lands 123, 133 may be arranged at opposite ends of the coupling portion 110. In other embodiments, the lands 123, 133 may be defined by oppositely directed faces of projections extending from respective parts 1230, 130.

Device 100 and other device embodiments described herein advantageously allow application of the device to the left or right arm. Where the inner profiles of the clamping two jaws are not symmetrical about the bridge portion, the device can be readily reversed in orientation to accommodate placement on either the left or right arm.

In order to maintain the device 100 in the clamped position, the device 100 has at least one retention mechanism 140. As shown in the Figures, a retention mechanism 140 may be disposed on opposite sides of the coupling or bridge portion 110. The one or more retention mechanisms 140 are configured to retain the device 100 in a compressed, clamped position once the jaws 120, 130 are moved toward each other. In particular, at least one retention mechanism 140 is configured to allow the device 100 to adopt one of a plurality of retention positions in which the coupling portion 110 is retrained from adopting an unclamped position.

Each retention mechanism 140 may be configured to adopt one of a plurality of discrete retention positions as the jaws 120, 130 are moved from an unclamped position to a clamped position. The specific discrete retention position adopted in the clamped position will depend on the size of the limb about which the device 100 is positioned as well as the degree of compressive force applied in manually driving the jaws 120, 130 toward each other.

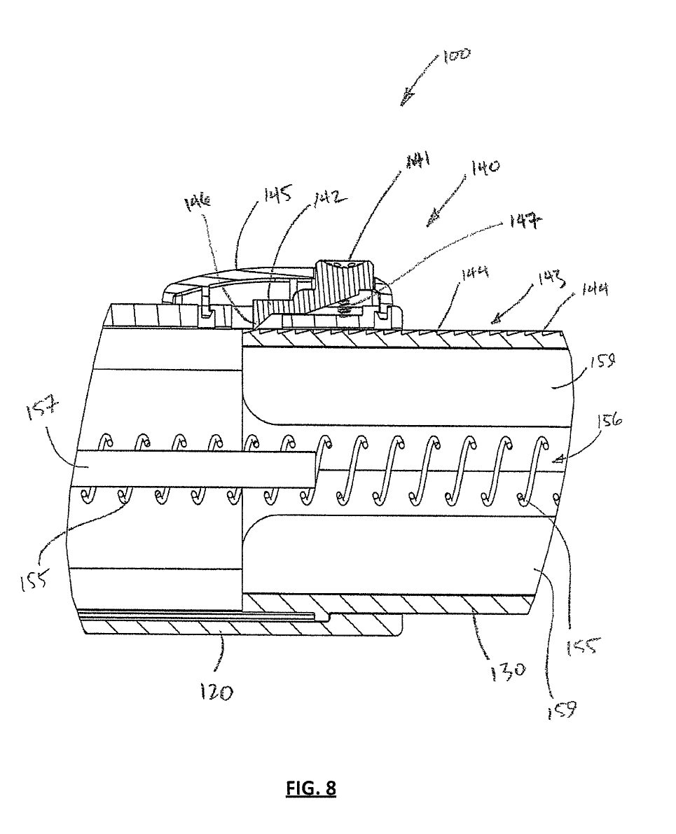

As shown in FIG. 2A and illustrated further in FIGS. 7 and 8, each retention mechanism 140 may comprise a ratcheting retention mechanism. This ratcheting retention mechanism 140 may comprise a linear rack 143 including a plurality of teeth 144 along which a pawl arm 142 travels between the clamped and unclamped positions. The pawl arm 142 comprises a manually releasable release actuator 141 at one end. The retention mechanism 140 comprises a biasing element 147 (FIG. 8) which cooperates with the pawl arm 142 to bias a pawl tooth 146 (on an opposite end of the pawl arm 142 from the release actuator 141) against the rack teeth 144. The pawl tooth 146 and rack teeth 144 are generally configured to permit relative movement of the pawl tooth 146 along the rack 143 in one direction, but tend to catch the pawl tooth 146 against one of the teeth 144 when a force might tend to cause relative movement in the opposite direction.

The pawl arm 142 further comprises a pawl pivot rocker 148 disposed intermediate the pawl tooth 146 and the release actuator 141 to allow the pawl arm 142 to pivot between an engaged position, in which the pawl tooth 146 catches against one of the teeth 144, and a release position, in which the pawl tooth 146 is free of interference from the teeth 144. The biasing element 147, which may be a spring for example, is positioned and configured to bias the pawl arm 142 toward the engaged position. A pawl cover 145 covers most of the pawl arm 142 from external interference, other than the release actuator 141, which is exposed for manual depression to move the pawl arm 142 to the release position. The pawl pivot rocker 148 may be received in a slightly recessed seat in (or otherwise held within the pawl cover 145 against) an external part of the first part 120.

The shape of the device 100 can be described as generally C-shaped or U-shaped, depending on the device orientation, featuring an opening between the opposed first and second distal portions 122, 132, with the bridge 110 at the apex opposite the opening. The space interior of the first and second jaws 120, 130 is thus generally concave to accommodate a limb and can be flattened as the jaws are pressed inwardly to close about the limb. At a minimum, at least one of the first inner profile 125 and the second inner profile 135 is generally non-linear. This non-linearity may take the form of a somewhat concave curvature along the respective jaw inner profile or a partially straight and partially curved profile. The first and/or second non-linear inner profile 125/135 may have two or more straight sections (angled relative to each other or separated by a curved section) and/or may have two or more sections of different curvature.

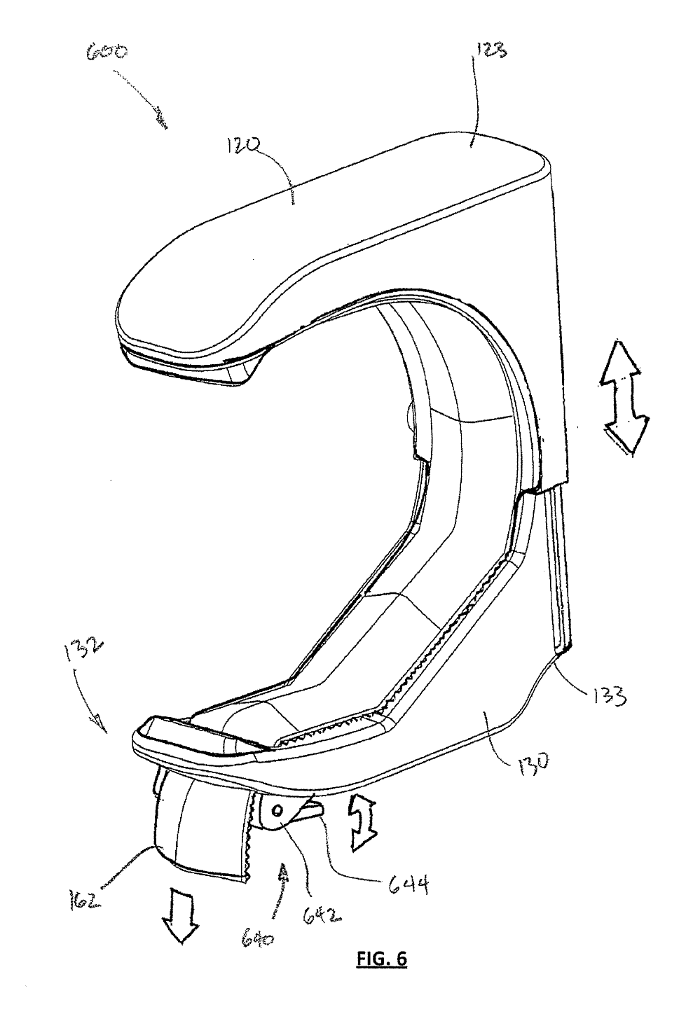

In the device embodiments 100 shown in FIGS. 2A to 5B, the first inner profile 125 is curved in a somewhat concave manner to be pressed against the medial surface of the limb 10 and the second inner profile 135 has a first slightly curved section 136 near the bridge apex (corresponding to the coupling portion 110), a generally straight section 137 that is angled relative to the curved section 136 and angled relative to a longitudinal axis of the bridge (along which relative movement occurs) and a second slightly curved section 138 that is angled relative to the straight section 137 and extends to the distal end 132. The second curved section 138 may be generally straight in some embodiments. The straight section 137 need not be perfectly straight. The purpose of the straight section 137 is to apply direct pressure to the cephalic vein 22 when the device 100 is placed over an upper arm 10, as is most clearly illustrated in FIG. 5B. The configuration of the second inner profile 135, including straight section 137, is therefore arranged to apply pressure generally inwardly to the lateral side of the upper arm 10 but in particular to the top or upper lateral part of the upper arm 10.

Along with pressure applied to the lateral side of the arm by the second inner profile 135, the first inner profile 125 applies inward pressure to inwardly compress the medial side of the upper arm 10. In order to effectively compress the basilic vein 24 and the brachial veins 26, the first inner profile preferably has a ridge, projection or bead 127 extending along the inner surface of the first jaw 120 between its distal end 122 and a point close to, but still somewhat distal of, the bridge portion 110. This ridge, projection or bead 127 serves to focus the pressure applied to the medial side of the arm and thereby more effectively compress the veins on the medial side of the upper arm 10.

Device 100 may have a cushioning element 160 that extends around (or mostly around) an inner periphery of the bridge 110 and first and second arms 120, 130. This cushioning element 160 may cover the ridge, projection or bead 127 on the first jaw 120 and may be affixed to the first jaw 120 at one more points. The cushioning element 160 may be freely slidable with respect to the second jaw 130 along the second inner profile 135 to allow for accommodating the relative movement between the first and second jaws during clamping and unclamping.

The second jaw 130 may have formed in its distal end 132 a passage 139 to receive an end portion 162 of the cushioning element 160. The passage allows travel of the cushioning element 160 through the distal end passage 139 as the cushioning element 160 moves along the second inner profile 135 during clamping or unclamping. This way, the end portion 162 does not interfere with the patient's arm during the clamping action and the cushioning element 160 experiences minimal or no bunching and there is minimal or no pinching of the arm by the cushioning element 160.

FIG. 2B illustrates an example cross-section of the cushioning element 160 along the length of it where it overlies the second inner profile 130. The cushioning element may have a backing layer 161 and a cushioning layer 164. Along at least part of the cushioning element 160, the backing layer 161 may have a length-wise bead 163 projecting inwardly (away from a flat backing section of the backing layer 161) toward the space 115. This bead 163 may provide a similar function to the ridge 127 in focussing compression on the arm.

Referring in particular to FIGS. 7 and 8, the internal structure of the coupling portion 110 is described in further detail. In order to bias the device 100 into the open position, a biasing element, for example in the form of a spring 155, is positioned to bias the first and second parts 120, 130 away from each other. In particular, the biasing element biases first and second portions of the coupling portion 110 away from each other. Since these first and second portions of the coupling portion 110 are attached to or integrally formed with the first and second parts 120, 130, the first and second parts 120, 130 are biased away from each other under the action of the biasing element.

The internal structure of the first and second parts 120, 130 is arranged to accommodate the biasing means extending longitudinally inside both the first part 120 and the second part 130 in a manner that may be generally parallel to a back or spine of the body 101 of the device 100. In order to retain the biasing element (in the form of spring 155) in position, a rod seat 158 is provided internally at one end of the device body 101 to receive one end of a bias element alignment rod 157. The other end of the bias element alignment rod 157 extends at least part-way into a cavity or chamber defined by the second part 130. The spring 155 is coiled around at least part of the bias element alignment rod 157 so that compression and extension of the spring 155 within the first part 120 generally occurs along the rod 157, as shown in FIGS. 7 and 8. The bias element alignment rod 157 may be sized to act as a stop to prevent relative movement of the first and second parts past a certain point, for example by abutting an internal structure of the second part.

The second part 130 defines an internal cavity or chamber with wall and/or flange structures 159 defining a spring channel 156 through which the spring 155 extends. Such wall/flange structures 159 serve an alignment function to keep the spring 155 generally longitudinally aligned during compression and extension within the second part 130.

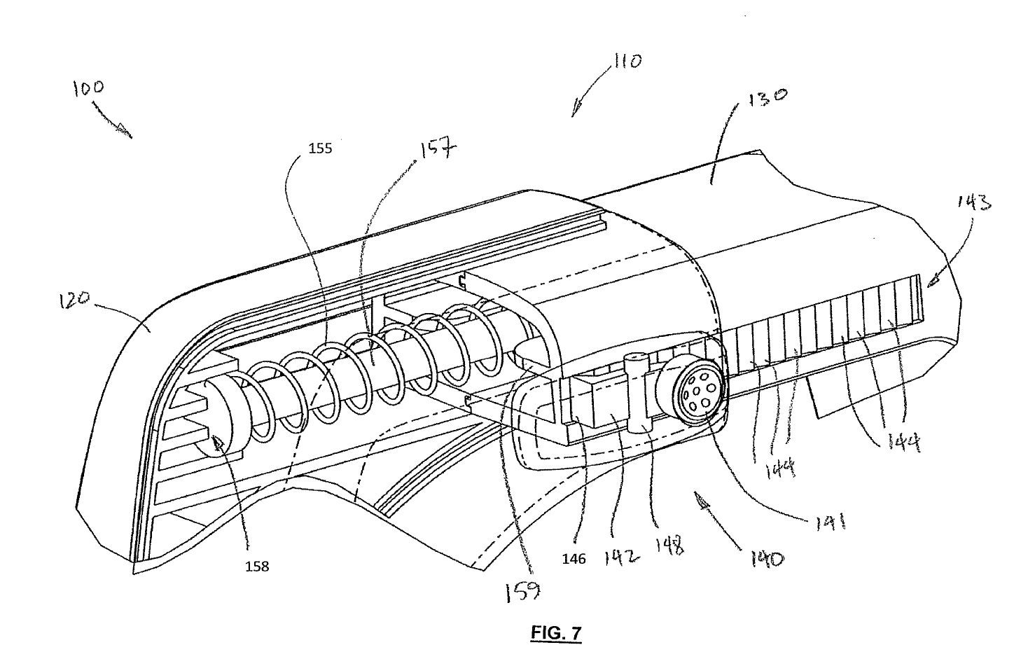

Referring now to FIG. 6, an alternative or additional retention mechanism 640 is shown, by which a clamping device 600 can be retained in a clamped position. The clamping device 600 may be identical to the clamping device 100, except that it uses the modified retention mechanism 640 as an alternative or addition to the retention mechanism 140 described in relation to device 100.

The retention mechanism 640 comprises a frictional retention mechanism that acts to frictionally engage the liner tongue 162 on an external side of the liner tongue passage 139. The retention mechanism 640 may comprise a spring biased lever 644 that pivots about a pivot axis that is generally parallel to the lateral extent of the liner tongue 162 and the external surface of the distal end 132 of device 600.

At least one anchor component 642 is formed or mounted on the external surface of the distal end 132 adjacent the external outlet side of the passage 139, so that when the anchor component 642 secures the lever 644 to rotate about the lateral pivot axis, a cam (formed on a part of the lever 644 adjacent where the liner tongue 162 projects) is arranged to impinge on and frictionally engage with the back side of the liner tongue 162 when the lever 644 is biased into a retaining position. The cam of the lever 644 is arranged so that the liner 160 can be pulled further through the passage 139 without significantly increasing the frictional engagement between the cam and the back of the liner 160, which serves to retain the device 600 in the clamped position.

Forces that would tend to push the device toward the unclamped position are resisted by increased frictional engagement of the cam (under the rotational spring biasing force of the lever 644) with the back of the liner 160 when the liner 160 is effectively pulled relative to the second part 130 toward an internal side of the second part 130. In order to release the retention mechanism 640, the lever 644 may be depressed, which moves the cam on the other end of the lever 644 away from a frictionally engaging position with the liner 160, thereby allowing the liner 160 to be easily withdrawn back through the passage 139 as the device opens from the clamped position to the unclamped position.

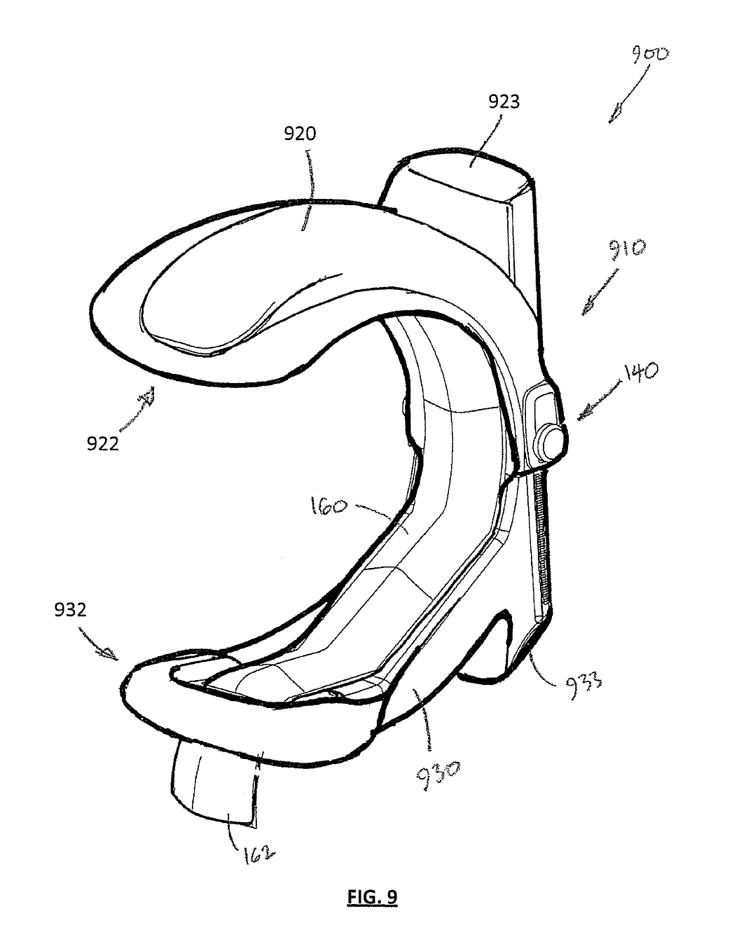

Referring now to FIG. 9, further clamping device embodiments are shown and described, in the form of an example clamping device 900. The clamping device 900 is quite similar to devices 100 and 600, but has accentuated curvature and padding around distal first and second ends 922, 932 of respective first and second parts 920, 930. A coupling or bridge portion 910 joins the first and second parts 920, 930 in a similar manner to devices 100 and 600, as described above. Additionally, device 900 is shaped to have easily recognisable and graspable lands 923 and 933 on opposite ends of the device 900 to readily allow manual compression of the device from an unclamped position (or a partially clamped position) to a fully (or more fully) clamped position by squeezing together of a person's thumb and fingers. It can thus be observed from FIG. 9 that the described embodiments of the clamping device need not be exactly in the form shown in FIGS. 1 to 8, but may accommodate some variation in shape and configuration while performing a similar function.

Referring now to FIG. 10, a clamping device 1000 according to further embodiments is shown and described. The clamping device 1000 is shown schematically for illustration purposes to have a bridge portion 1010 to which are rotatably coupled first and second movable jaws 1020 and 1030. The jaws 1020, 1030 are independently movable relative to each other and to the bridge portion 1010 so that they can adopt an open position in order for the device 1000 to be placed about a portion of a limb, such as an upper arm, and a closed or clamped position, where the device 1000 has the first and second jaws 1020, 1030 pressing against the limb, such as on medial and lateral sides of the limb.

In a generally analogous form to the clamping devices 100, 600 and 900 shown and described herein, the clamping device 1000 employs a form of retention mechanism to retain the device 1000 in the clamped position. In the illustrated embodiments of clamping device 1000, first and second retention mechanisms 1042 and 1043 are employed, for example, in the form of respective ratcheting retention mechanisms that allow progressive rotation of each of the first and second jaws 1020, 1030 in the clamped position but resist movement back into the unclamped position unless a manually actuable release mechanism, such as a depressible button (not shown) is actuated.

Similarly to the clamping device embodiments described above, the first and second jaws 1020, 1030 of clamping device 1000 may have a non-linear inner profile where those jaws are arranged to impinge on the surface of the limb to be clamped. This non-linear profile may be provided on one or both of the inner faces of the first and second jaws 1020, 1030. The non-linear profiles of the rigid jaws 1020, 1030 may take the form of a generally concave surface (optionally with a projecting ridge analogous to ridges 127 and 163). Alternatively, one or both of the inner profiles of the rigid jaws 1020, 1030 may have a series of straight portions angled with respect to each other to in effect define a roughly concave inner profile. Alternatively, the inner profile may have at least one curved section and at least one straight portion.

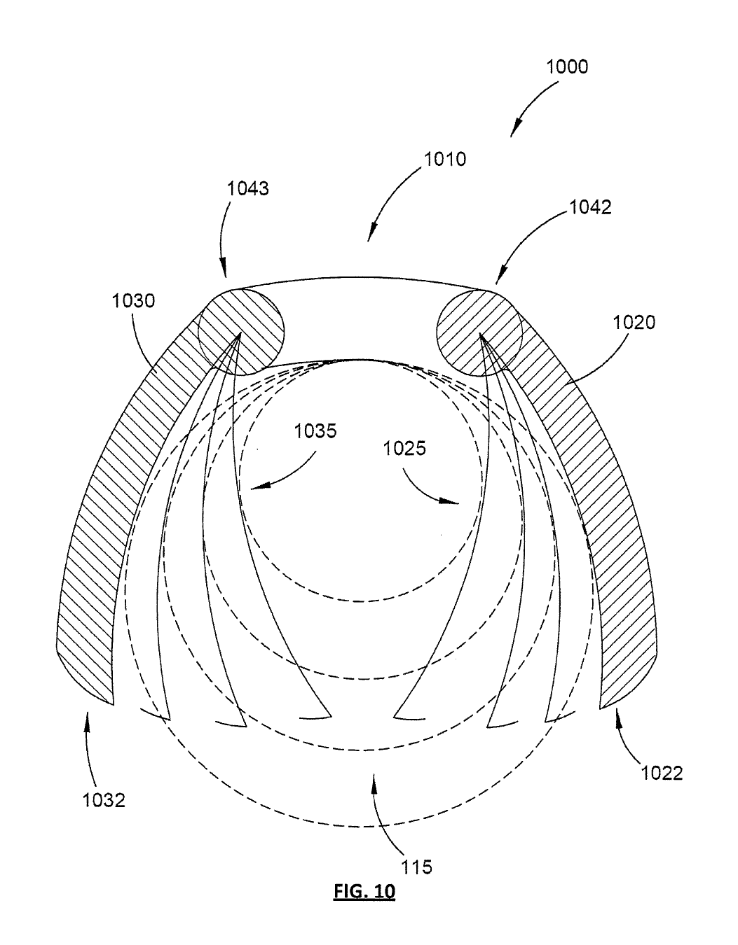

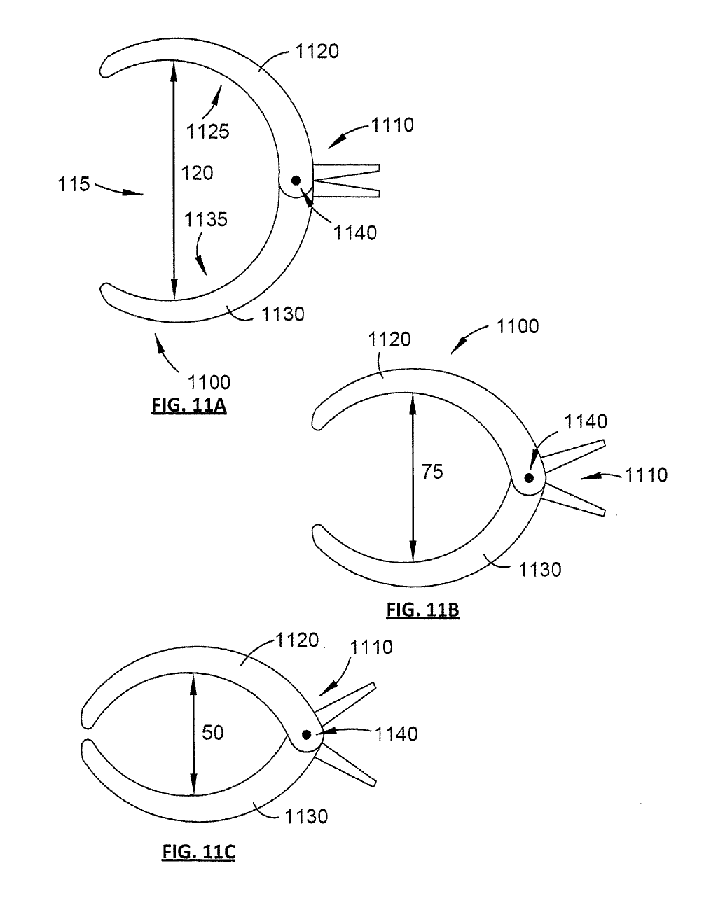

Referring now to FIGS. 11A, 11B and 11C, a clamping device 1100 according to further embodiments is described. Clamping device 1100 has a bridge or coupling portion 1110 that defines a pivot axis about which first and second opposed jaws 1120, 1130 may rotate relative to each other when moving between a clamped position and an unclamped position. As with other embodiments described herein, the opposed first and second jaws 1120, 1130 define a space 115 therebetween in the unclamped or open position in order to allow the device 1100 to be placed about a limb. The first and second inner profiles 1125, 1135 of the respective first and second jaws 1120, 1130 are shown as being generally curved in a concave form, although the concave form may be achieved by including one or more straight portions and/or more than one curved section in order to provide more targeted compression of particular veins in the limb to be clamped.

The clamping device 1100 may employ a retention mechanism 1140 that includes a rotationally ratcheting retention mechanism. Although not shown, a manually actuable release mechanism, such as a depressible button, may be used to allow the device 1100 to adopt the unclamped position (from the clamped position) under the action of a suitable biasing element, such as a spring (not shown).

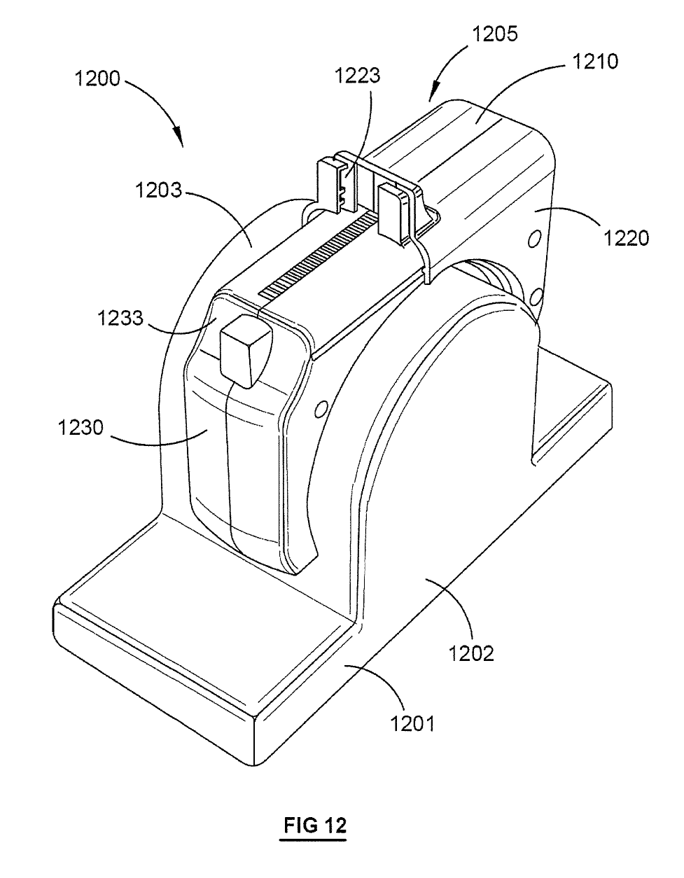

According to some embodiments, the clamping device may be accompanied by kit components that may assist in hygienic storage and use of such clamping devices. As shown in FIG. 12, for example, a saddle device 1201 may be provided as part of a kit 1200 that also includes a clamping device 1205 according to still further embodiments. The saddle 1201 may comprise a generally horizontal base portion 1202 for placement on a flat surface and a generally upwardly projecting convex portion 1203 of a size and shape to generally match and fit within the space 115 defined between first and second jaws 1220, 1230 of the device 1205 when it is in the open unclamped position.

The clamping device 1205 shown in FIG. 12 may define oppositely directed lands 1223 and 1233 on respective first and second parts 1220, 1230 that are not at opposite ends of the device body but are instead positioned more closely together along a spine region 1210 (analogous to the bridge coupling portion of the clamping device embodiments described above). Thus, the lands used to manually compress the device into the clamped position need not necessarily be located at opposite ends of the clamping device but may instead be defined by one or more projecting portions that are of suitable orientation for manual engagement of a thumb and fingers in a squeezing action.

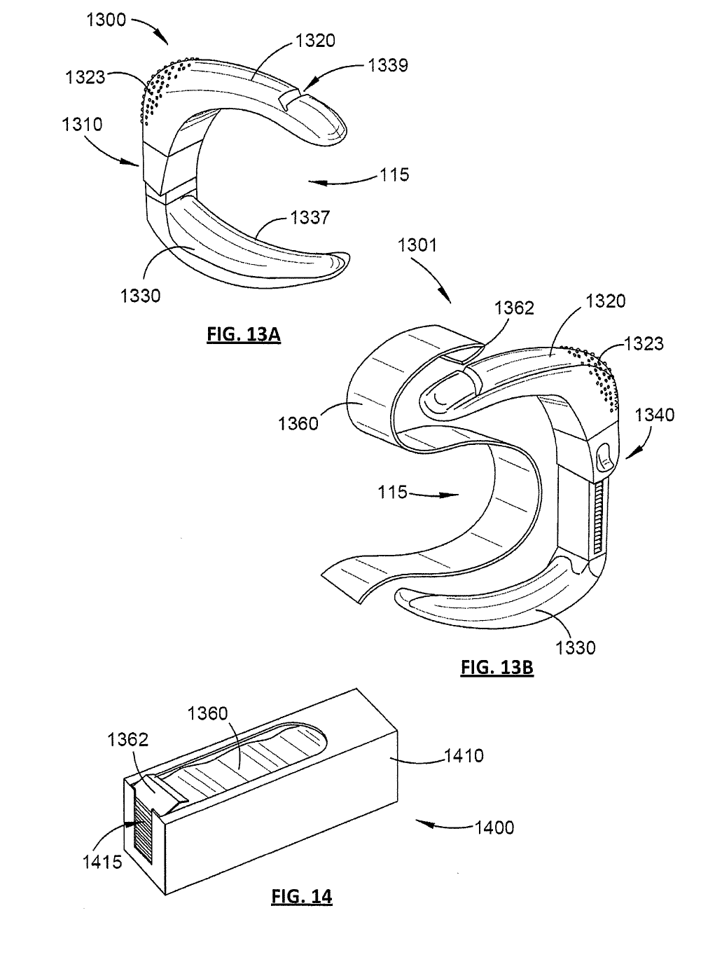

Referring now to FIGS. 13A, 13B and 14, a clamping device 1300 according to further embodiments is shown and described, as part of a kit comprising a disposable clamp liner 1360. The kit may comprise multiple such disposable liners 1360 contained in a suitable container 1400, for example.

The clamping device 1300 functions in a generally similar manner to the clamping device embodiments described above in that it has a bridge or coupling portion 1310 that joins opposed first and second jaws 1320, 1330 in a manner that allows them to move between a clamped position and an unclamped position. A retention mechanism 1340 (in this case a linear ratcheting mechanism with a release actuator similar to that described and shown in relation to FIGS. 1 to 5, 7 and 8) is used to retain the device 1300 in the clamped position.

Clamping device 1300 may have an elastomeric over-mould 1323 formed or fitted onto or around most of the device body, including the first and second generally rigid jaws 1320, 1330. This over-mould 1323 may comprise a material with a relatively high co-efficient of friction with respect to human skin so as to improve gripability (frictional characteristics) of the device 1300 when applying the device 1300 to clamp a limb. Suitable compression ridges may be formed to project from one or both of the inner profiles of the first and second jaws 1320, 1330.

A recess or detent 1339 may be formed in an external surface toward a distal end of one of the first and second jaws 1320, 1330. This recess or detent 1339 is shown in FIG. 13A and in FIG. 13B by way of example as being in an external surface of the first jaw 1320. This recess or detent 1339 is sized and arranged to receive a folded end portion 1362 of one of the flexible disposable liners 1360, so that the remainder of the liner 1360 can be pressed or folded into the space 115 and generally overly the U-shaped inner profile defined by the opposed first and second jaws 1320, 1330 in the clamped and unclamped positions. The end of the disposable liner 1360 that is not received in the recess or detent 1339 is left as a free end so that movement of the liner 1360 at its free end is allowed in order to mitigate pinching of the limb as the device 1300 moves towards the clamped position.

In order to at least partially temporarily affix the disposable liner 1360 to the device 1300, an adhesive substance may be provided on a back surface (opposite to a limb engaging surface of the liner 1360) to adhere the liner 1360 to the same jaw (eg, the first jaw 1320) which retains the one end 1362 of the disposable liner 1360. This adhesive substance may be exposed by removal of a peel away backing sheet, for example, and should not be so strong as to make manual removal of the liner 1360 from the device 1300 difficult.

The container 1400 of disposable liners 1360 may include a number of such liners 1360 in a ready to remove form, so that each liner can be removed from a body 1410 of the container 1400 through a top and/or side aperture 1415. The disposable liners 1360 may be arranged in the container 1400 in a manner that allows the one end 1362 (to be received in the recess or detent 1339) to be readily identified and used as a means to withdraw the disposable liner 1360 from the container 1400.

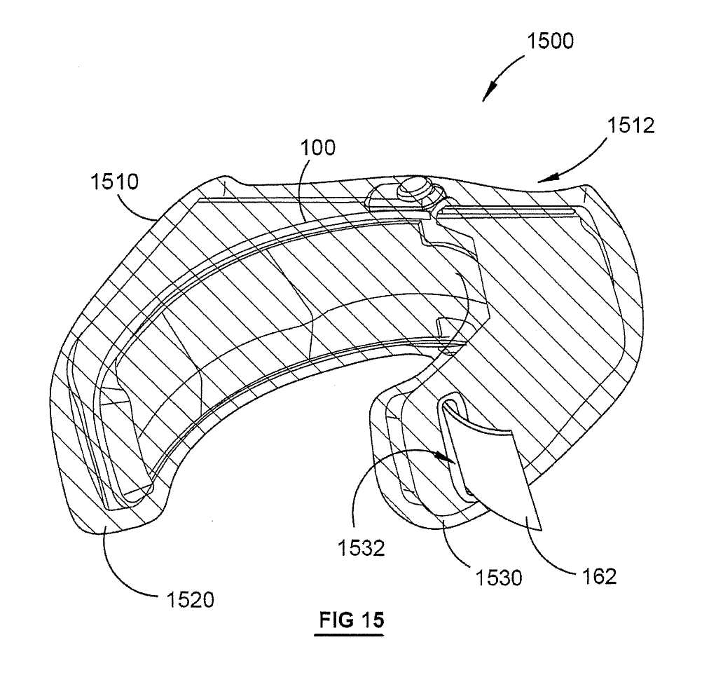

Referring now to FIG. 15, a further form of disposable liner 1510 is shown and described as part of a kit 1500 that also includes the clamping device 100 (as one example of various described clamping devices usable with the disposable liner 1510). The disposable liner 1510 may be roughly in the form of a flexible plastic sack that substantially covers most of the body of the device 100, but for an opening 1512 along the backside or spine of the device 100. The opening 1512 exposes an opposite face of the device 1300 to the inner apex of the U-shape defined by the first and second parts 120, 130.

The disposable liner 1510 defines opposed pouch portions 1520 and 1530 for receiving and substantially enclosing the opposed first and second portions 120, 130, respectively, with a bridging section that joins the two pouch portions 1520, 1530. In this way, the entire inner U-shaped clamping profile of the device 100 is covered by the disposable liner 1510, while the liner 1510 remains easily pulled off the device 100 by withdrawing the device 100 from the sack through the opening 1512 in the back of the liner 1510. Optionally, the second pouch portion 1530 may have a slit or aperture 1532 formed in an outer side thereof to allow the liner tongue 162 to extend therethrough.

The disposable liner 1510 is preferably formed of a hygienic plastic or fabric material that is easy to put on and taken off and does not impede or interfere with the clamping and unclamping actions of the clamping device 100 (or other embodiments described and depicted herein).

Kit 1500 may comprise multiple disposable liners 1510 arranged in a stack so as to be sequentially opened out and pulled off the stack as each successive disposable liner 1510 is used. Although not shown, preferably the kit 1500 includes a holding device, similar to those currently known and in use for plastic shopping bags and umbrella sleeves, to hold the stack of disposable liners 1510 so that, as the clamping device 100 (or any other clamping device embodiments described herein) is inserted with its jaws into the pouches 1520, 1530 of the disposable liner 1510 and pulled away from the rest of the stack of such liners, another liner is pulled into a position ready for similar use.

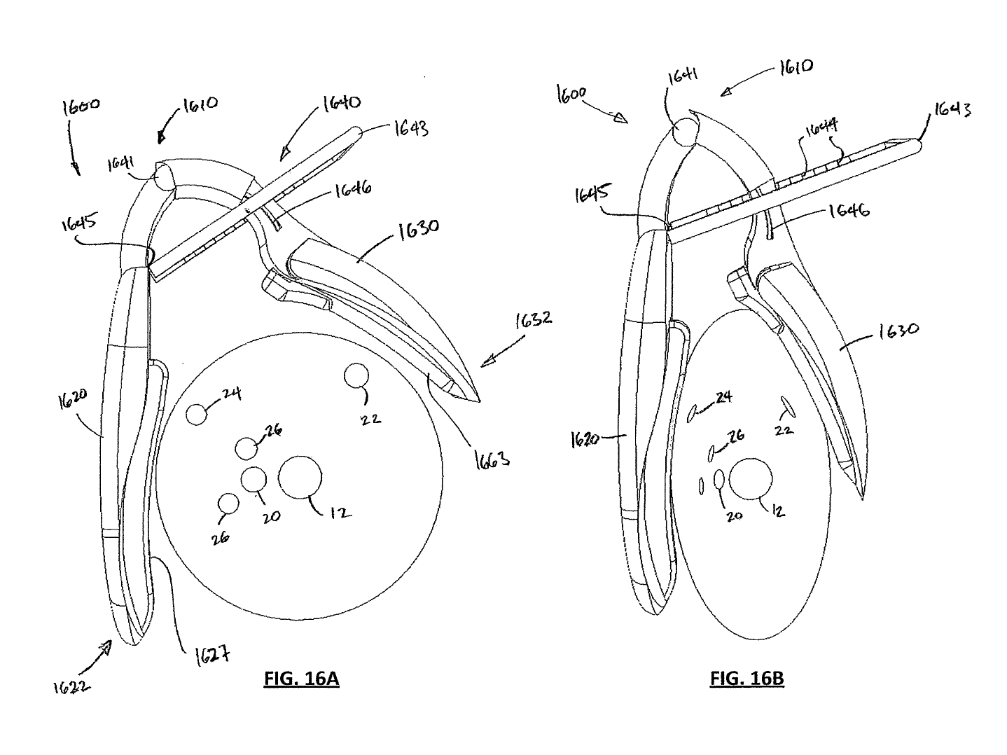

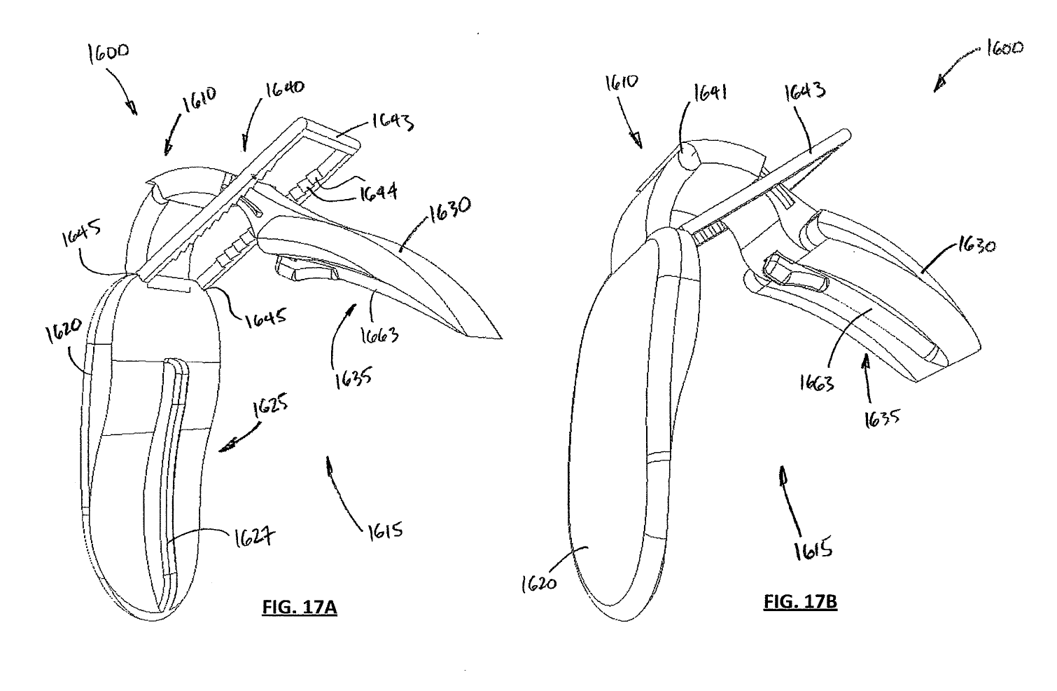

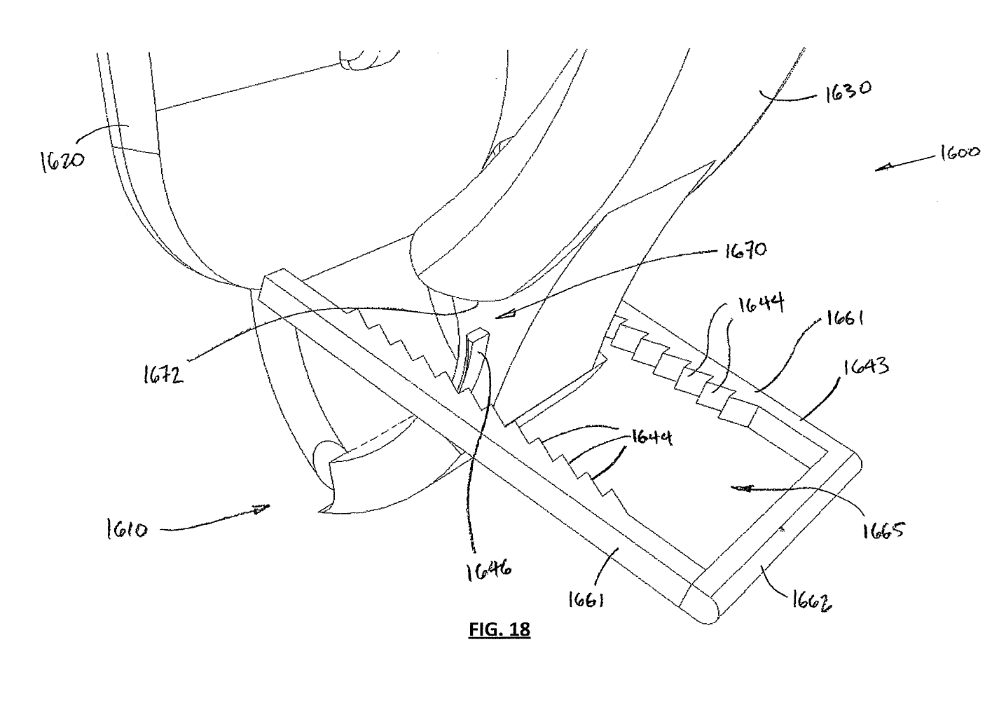

Referring now to FIGS. 16A, 16B, 17A, 17B, 18, 19 and 20, a clamping device 1600 according to some further embodiments is shown and described. The clamping device 1600 operates on similar principles to the clamping devices described above and includes a coupling or bridge portion 1610 that couples a first jaw 1620 with a second jaw 1630 in a manner that allows relative movement of the first and second jaws 1620, 1630 between clamped and unclamped positions.

The clamping device 1600 has a retention mechanism 1640 to retain the device 1600 in the clamped position. This retention mechanism 1640 may be a ratcheting retention mechanism, for example. The ratcheting retention mechanism may comprise a loop 1643 that has ratchet teeth 1644 linearly disposed to progressively engage with at least one lateral pawl projection 1646 on one of the first and second jaws 1620, 1630. The loop 1643 is anchored at an anchor point 1645 adjacent a shoulder of the other one of the first and second jaws 1620, 1630 in the example illustrated. The loop 1643 is anchored at the anchor point 1645 adjacent a shoulder of the first jaw 1620 in a manner that allows pivoting of the loop 1643 about the anchor point 1645. This allows the loop 1643 to move with the second jaw 1630 as it rotates about a rotatable coupling 1641 that connects the first and second jaws 1620, 1630 and forms part of coupling portion 1610.

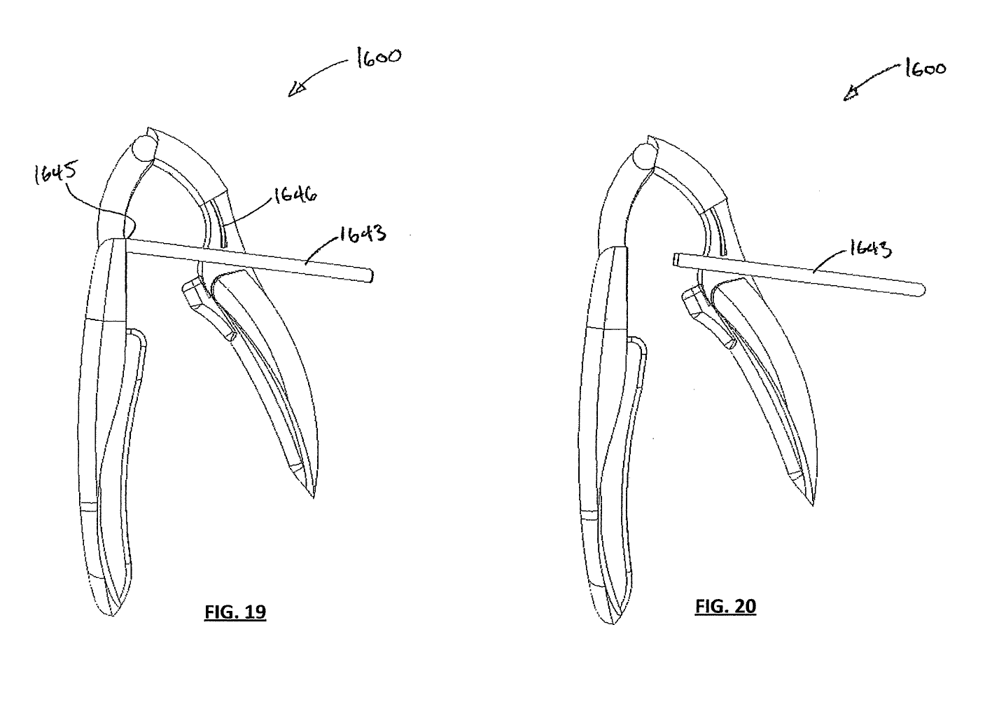

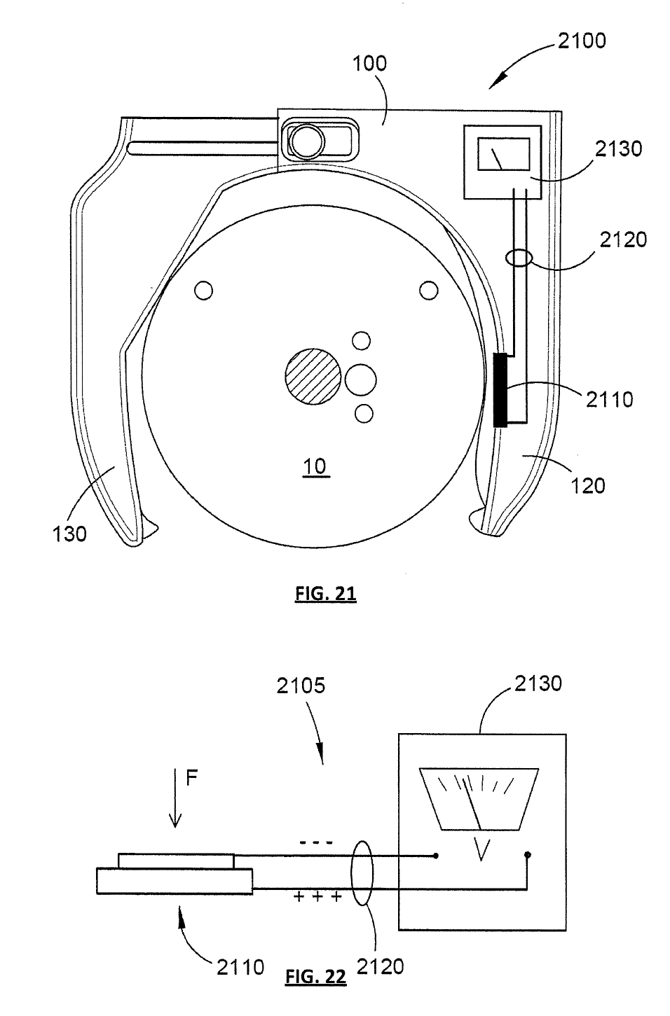

The shape of the pawl projection 1646 is most visible in FIG. 18. In order to allow the clamping device 1600 to return to the unclamped position from the clamped position, the loop 1643 can be pivoted to a release position beyond one edge of the pall projections 1646 so that the teeth 1644 no longer engage with the pawl projections 1646. In this release position, the loop 1643 aligns with a gap 1670 formed in between one end of the pawl projections 1646 and an adjacent shoulder 1672 of the second jaw 1630. This is illustrated in FIG. 19. Alternatively or additionally, the loop 1643 may be connected to the first jaw 1620 in a manner that allows the loop 1643 to be broken away or detached from the first jaw 1620 at the anchor point 1645 or another part of the loop 1643, as illustrated in FIG. 20. In such embodiments, where the device 1600 is intended to only allow a single use, the gap 1670 may not be present and the frangible attachment of part or all of the loop 1643 to the first jaw 1620 may assist in ensuring that the device 1600 is not used multiple times, since the broken loop 1643 would not operate to retain the device 1600 in the clamped position.

As shown best in FIG. 18, the loop 1643 comprises opposed linear sets of ratcheting teeth 1644 arranged on generally parallel linear ratchet arms 1661, which are joined at an outer apex of the loop 1643 by an end loop or bar 1662.

As is evident from the drawings, some clamping device embodiments shown and described herein are asymmetrical about the apex of the U-shape of the device, such that one jaw may be differently shaped and sized from the other jaw. This may in some embodiments assist targeted compression of certain veins in the limb to be compressed.

As shown in FIGS. 16A to 20, clamping device 1600 is generally asymmetrical, with the first jaw 1620 being longer than the second jaw 1630 and configured to press against a medial side of an upper arm, as illustrated in FIG. 16B. On the other hand, the second jaw 1630 is arranged to be pressed against an upper lateral part of the upper arm in order to target compression of the cephalic vein 22 while the first jaw 1620 targets compression of the basilic and brachial veins 24 and 26.

Thus, the clamping device 1600 is sized and arranged such that, when the limb is an upper arm and the device 1600 is placed in the clamped position about the upper arm with one of the first and second jaws 1620, 1630 pressing against a medial side of the upper arm, the other of the first and second jaws 1620, 1630 presses against an upper lateral part of the upper arm to compress the cephalic vein.

As shown in FIGS. 16A to 20, device 1600 has a first longitudinal ridge 1627 projecting from an inner profile of the first jaw 1620 in order to target compression of the veins on the medial side of the limb, while the second jaw 1630 has a ridge 1663 positioned to target compression of the cephalic vein 22 on a top lateral side of the upper arm.

As illustrated in the Figures, most of the clamping device embodiments are not intended to have the distal ends of the jaws touch each other when in the clamped position. Thus, at least some of the described clamping device embodiments are generally not arranged to entirely encircle the limb, nor are they arranged to contact and compress the entire circumference or periphery of the limb.

As shown in FIGS. 16A to 20, the clamping device 1600 has generally slightly concavely curved inner profiles where the first and second jaws 1620, 1630 are to contact the limb. In alternative embodiments, such first and second inner profiles may have portions of different curvature and/or generally straight sections.

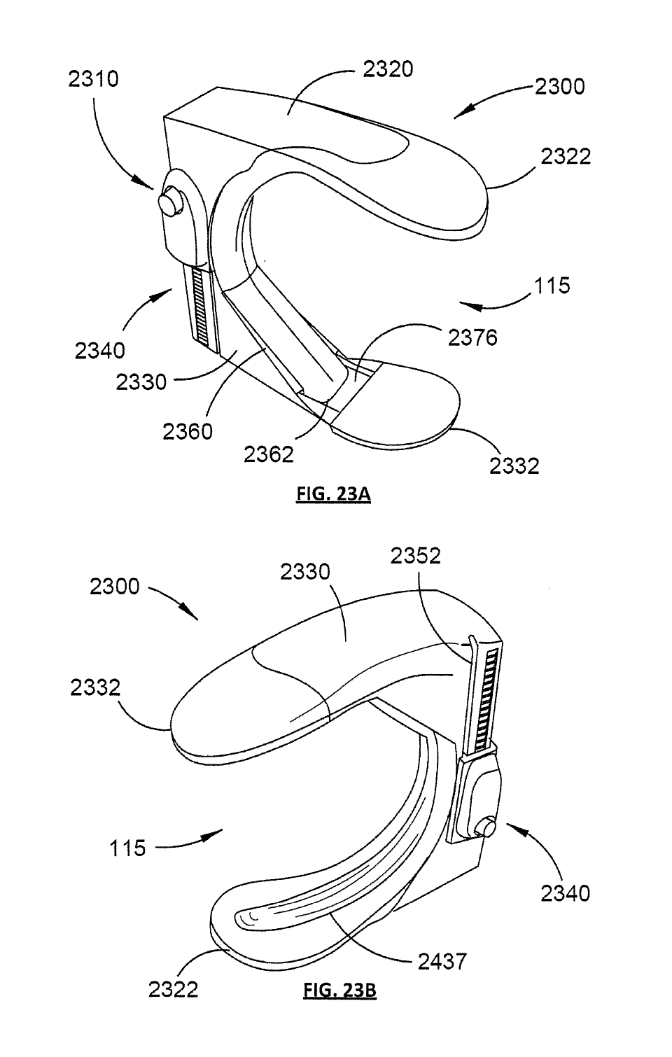

Referring now to FIGS. 21 and 22, an example clamping device 2100 is shown, including a pressure sensor 2105 arranged to sense pressure on a part of the inner profile or inner face of one or both of the first and second jaws 120, 130. The clamping device 2100 may be the same as device 100, but for the inclusion of the pressure sensor 2105. Alternatively, the pressure sensor 2105 may be incorporated within other clamping device embodiments described herein and suitably arranged to sense a clamping pressure applied to a limb when the clamping device is in a clamped position on the limb.

The pressure sensor 2015 comprises a pressure transducer element 2110, which can be a piezoelectric element, for example. The pressure transducer element 2110 may be electrically coupled via suitable insulated conductors 2120 to provide an output signal indicative of the sensed pressure to a display 2130. The display 2130 is arranged to indicate (in response to the received output signal) the pressure sensed by the transducer element 2110, so that a person, such as a medical practitioner, can readily view the display 2130 and ascertain whether the clamping device 2100 has been applied with too much compression, not enough compression or a degree of compression that is appropriate.

Although the pressure sensor 2105 is shown in FIG. 21 as being disposed in the first part or jaw 120, with the pressure transducer element 2110 (such as a pressure sensor) being positioned adjacent the inner face or ridge extending along the inside of the first part or jaw 120, the pressure transducer element 2110 may be positioned at a different position around the inner profile of the clamping device 2100. Additionally, more than one pressure transducer element 2110 may be positioned around the inner profile of the clamping device 2100, either coupled via additional conductors 2120 to the same display 2130 or as part of multiple separate pressure sensors 2105.

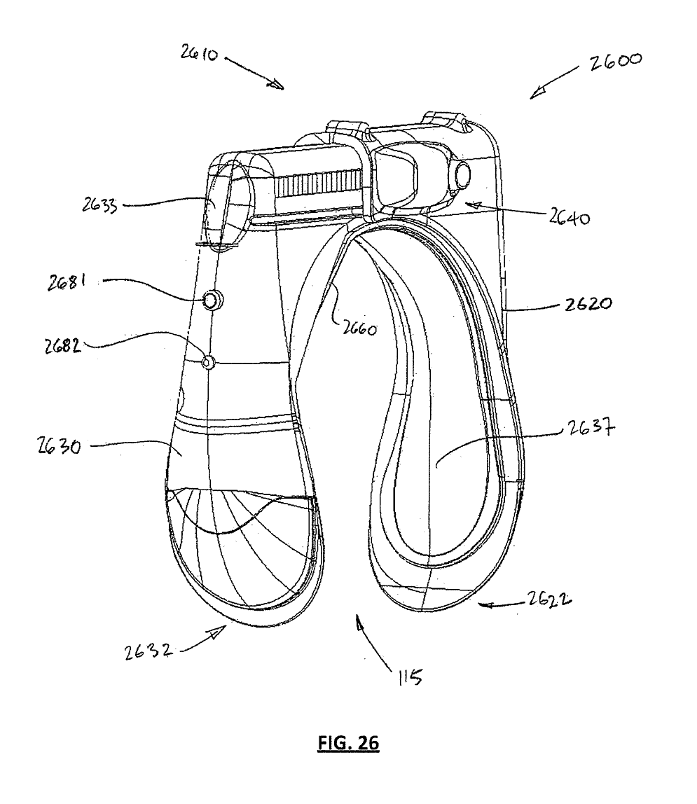

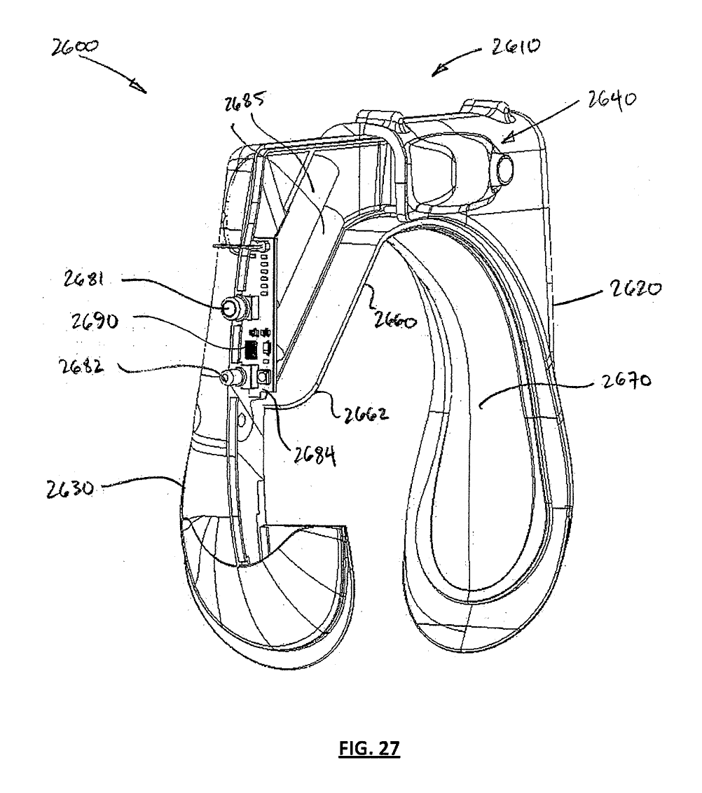

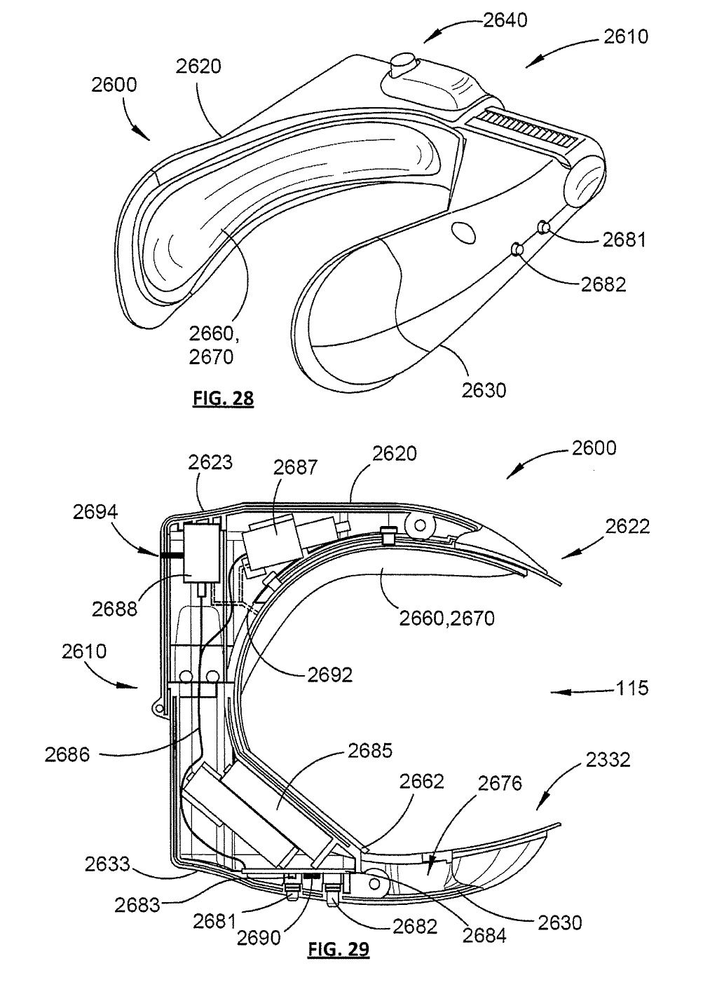

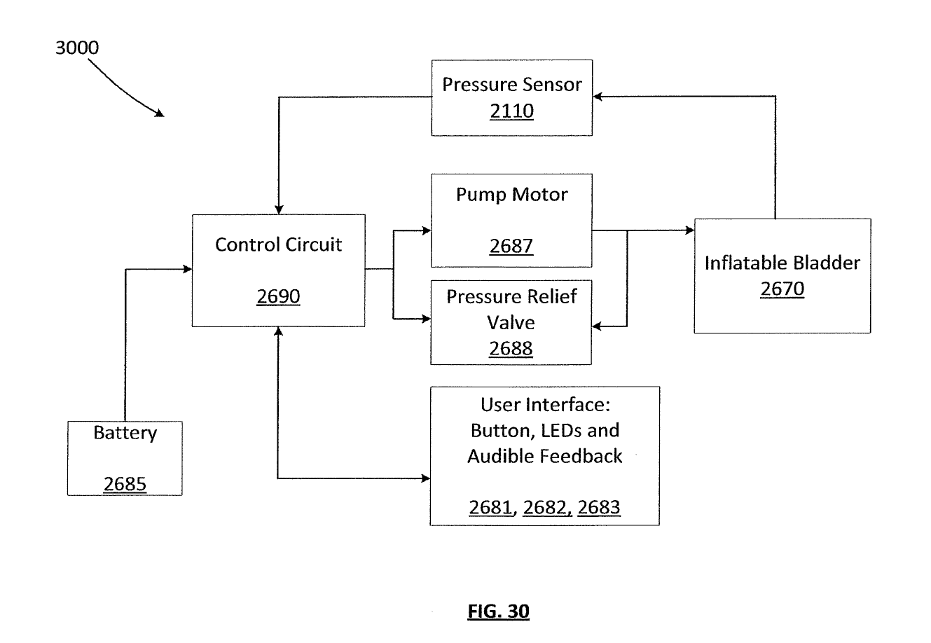

Referring now to FIGS. 24 and 25, an example expandable element is schematically illustrated in conjunction with a sphygmomanometer 2410, although other forms of blood pressure meter may be employed.

As shown in FIGS. 24 and 25, an expandable element 2470 may form part of a modified cushioning element 2460 similar to the example cushioning elements previously described, but with an inflatable part in the form of expandable element 2470. The expandable element 2470 may be formed as an inflatable portion of a cushioning element 2460, with the inflatable portion being defined by a bladder at least partially received within a substantial lengthwise part of the cushioning element 2460. Alternatively, the expandable element 2470 may be formed as a separate part from the cushioning element 2460, even though the expandable element 2470 may be co-located and at least partially co-extensive therewith.

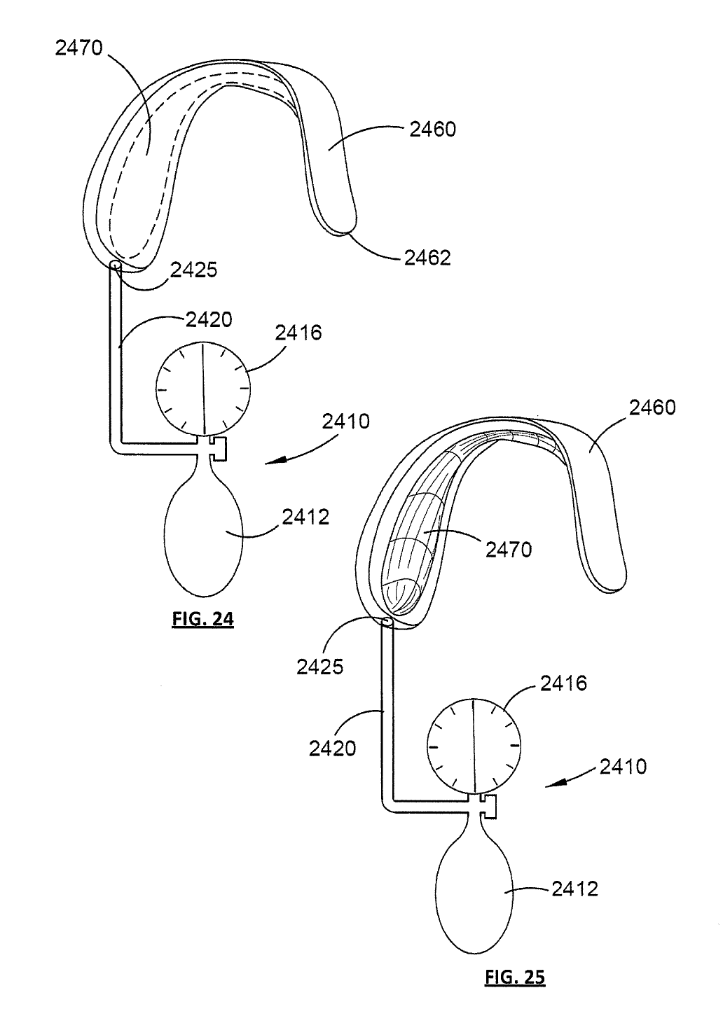

The expandable element 2470 can be used to couple with the sphygmomanometer 2410, for example via a suitable coupling valve 2425 at an exposed end of the cushioning element 2460 (for example at the end portion 2322 of the first jaw 2320 shown in FIGS. 23A and 23B). The sphygmomanometer 2410 may be an existing device, for example including a hand actuable pump 2412, a pressure indicator 2416 and a fluid conduit 2420 to pump air into and release air from the expandable element 2470 via the valve 2425.

The sphygmomanometer 2410 may also be an existing electronic sphygmomanometer that is capable of obtaining blood pressure measurements in an autonomous manner once the clamping device the sphygmomanometer is coupled to has been correctly positioned and clamped onto the arm and measurement has been initiated (for example by depressing a button or switch).

The expandable element 2470 may form part of a core of the cushioning element 2460 and may extend at least partially along the longitudinal axis of the cushioning element 2460, optionally all the way to the tongue portion 2462 at the end of the cushioning element 2460, but possibly extending only in the order of a ½ to ⅘ of the length of the cushioning element 2460. Advantageously, the pressure applied by the expandable element 2470 may be measured by the pressure sensor shown and described above in relation to FIGS. 21 and 22.

In an embodiment, device 2600 is provided with an expandable element 2670 within, under or co-located with a cushioning element 2660. The expandable element 2670 may comprise an inflatable bladder for applying pressure to at least part of an arm, for example when used to clamp the arm or when used to take blood pressure measurements. In some embodiments, expandable element 2670 is the same as expandable element 2470 and device 2600 may be coupled to the sphygmomanometer 2410.

In some embodiments, device 2600 further comprises control functions to automate the inflation and deflation of the expandable element 2670. Such control functions may be provided by a controller 2690 on a circuit board 2684 that is electrically coupled to a pump 2687 to operate the pump 2687 to pump air into (inflate) the expandable element 2670.

The controller 2690 is also electrically coupled to a pressure relief valve 2688 (for example in the form of a small solenoid valve) to control operation of the pressure relief valve 2688 and thereby selectively allow deflation of the expandable element 2670. The controller 2690 may control operation of the pressure relief valve 2688 to selectively allow progressive and/or staged deflation of the expandable element 2670, for example in a set or programmed manner that allows blood pressure measurements to be taken using the pressure sensor 2110. The pump 2687 may be positioned inside either the first part 2620 or the second part 2630, although in the illustrated embodiment, the pump is disposed in the first part 2620. A suitable air inlet 2694 may be provided in an external wall of the part of the device 2600 within which the pump 2687 is housed. The air inlet 2694 may also function as an air outlet during deflation or there may be a separate air outlet provided in an external wall of the device 2600.

Manually actuable input components may be positioned on an outside of one of the first part 2620 and the second part 2630 and can be used to provide user control input to the controller 2690, for example via inflation and deflation actuators 2681 and 2682. The inflation actuator 2681, which may be formed as a button, and a deflation actuator 2682, which may be formed as a deflation button, may be coupled to the circuit board 2684 and may be arranged to interact with the controller 2690 to cause the controller 2690 to send control signals via separate electrical conductors (wires) 2686 to the pump 2687 and the pressure relief valve 2688, respectively, to cause inflation or deflation of the expandable element 2670.

The inflation and deflation actuators 2681, 2682 may be positioned close to each other on an external (outwardly facing) wall of the second part 2630, in between a land 2633 (for applying manual force to bring the device 2600 toward a clamped position) and a distal end 2632 of the second part 2630, as is shown in the Figures. Alternatively, the inflation and deflation actuators 2681, 2682 may be positioned close to each other on an external (outwardly facing) wall of the first part 2620, in between a land 2623 (for applying manual force to bring the device 2600 toward a clamped position) and a distal end 2622 of the first part 2630. In either case, it is preferred that the inflation and deflation actuators 2681, 2682 are positioned on a part of the device 2600 that is away from the bridge portion 2610. In a further alternative, the inflation and deflation actuators 2681, 2682 may be positioned on a part of the bridge portion 2610 that does not interfere with relative movement between the first and second parts 2620, 2630 and does not interfere with the clamping or unclamping functions of the device 2600.

Inflation of the expandable element 2670 by the pump 2687 may be controlled to achieve an internal pressure of the expandable element at a first pressure set-point or at a second pressure set-point that is higher than the first set-point. The first pressure set-point may be a pressure from about 40 mmHg to about 80 mmHg, and optionally about 60-70 mmHg. The second pressure set-point may be a pressure from about 80 mmHg to about 200 mmHg, and optionally about 90-100 mmHg.

The pressure relief valve 2688 may be in communication with an air inlet/outlet 2694 in an external wall of the first part 2620 (for embodiments where the pressure relief valve 2688 and the pump 2687 are in the first part 2620). The pump 2687 may in some embodiments be in fluid communication with a separate air inlet/outlet (not shown) to inlet/outlet 2694. Small tubing 2692 may be provided inside the device housing to pneumatically couple the pump 2687, the expandable element 2670 and the relief valve 2688 so that air can be pumped into the expandable element 2670 and released therefrom via the relief valve 2688. A pressure sensor may also be pneumatically coupled to the pump 2687.

Optionally, one or more indicators or coloured lights, such as light emitting diodes 2683, may also be provided on (or otherwise coupled to) the circuit board 2684 and may be associated with each of the actuators 2681, 2682. When one of the actuators 2681, 2682 has been manually actuated, the controller 2690 may cause one or more of the LEDs 2683 to light up to visually indicate that inflation or deflation is occurring or is about to occur or to indicate a particular status of operation of the device 2600. In some embodiments (described below) where the inflation or deflation can be effected automatically through voice commands or externally originating control commands, the LEDs 2683 may be used to indicate the operational status (e.g. mid-level inflation, maximum level inflation, deflation or progressive (staged) deflation) of the expandable element 2670.

One or more batteries 2685 housed within the second part 2630 may provide power for the controller 2690, the circuit board 2684, the LEDs 2683, the pump 2687, the relief valve 2688, plus any other external communication function, such as an audible alarm or a wireless communication function. Although not shown, terminals of the one or more batteries 2685 are electrically coupled to the circuit board 2684 to provide a power source for the circuit board 2684. The other powered components, such as the LEDs 2683, relief valve 2688, pump 2687 and controller 2690, may receive power from the one or more batteries 2685 directly or via the circuit board 2684.

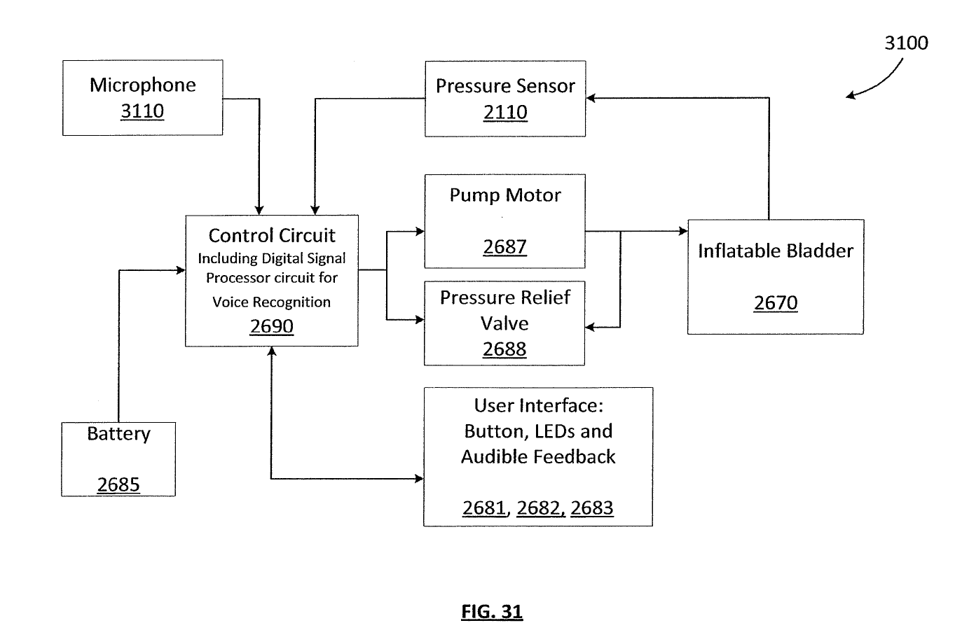

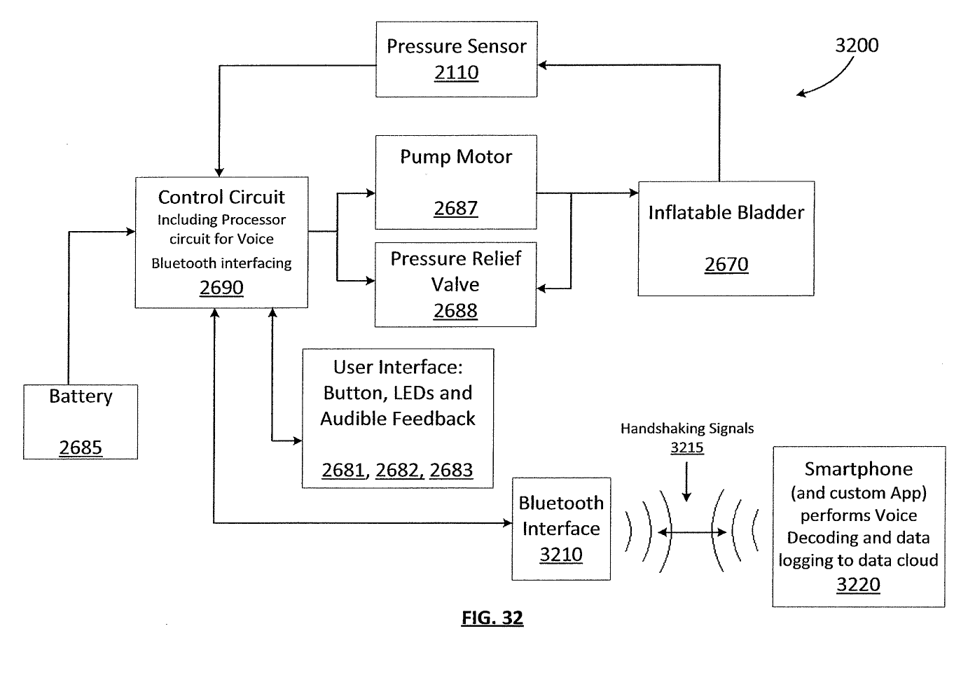

Optionally, the pressure sensor 2110 may be used to sense the pressure in the expandable element 2670 and to provide an output signal to the controller 2690 indicative of the sensed pressure. Alternatively, the pump 2687 may have a pressure sensing function and may provide an output signal to the controller 2690 indicative of the sensed pressure in the expandable element 2670. With such signals providing feedback, the controller 2690 can relatively accurately cause the expandable element 2670 to reach the desired pressure set-point. FIG. 30 is a schematic block diagram of a clamping device 3000 having the features and functions of the clamping device 2600 described above, plus the pressure sensor 2110 (or an equivalent sensor provided by the pump 2687). In some embodiments, a voice activation function may be provided to allow the inflation or deflation of the clamping device 2600 to be effected by voice commands. In such embodiments, schematically represented as device 3100 in FIG. 31, the device 2600 described above may be supplemented with an audio input component 3110, such as a microphone, and a digital signal processor (DSP) as part of the controller 2690. The controller 2690 in such embodiments may comprise suitable hardware and execute suitable software to process speech signals received via the audio input component 3110 to determine whether a valid voice command has been received. If a valid voice command is determined by the controller 2690 to have been received, then the controller 2690 operates the pump 2687 for inflation or the pressure relief valve 2688 for deflation (as appropriate) in response to the valid voice command. A trigger phrase may be used to prime the receipt of a following voice command. For example, the trigger phrase may be “command”, followed by an actual voice command, such as “deflate” or “inflate”. If no valid voice command is determined to have been received, no action is taken by the controller 2690.

In some embodiments, a remote device control function may be provided to allow the inflation or deflation of the clamping device 2600 to be effected by use of an external control device. In such embodiments, schematically represented as device 3200 in FIG. 32, the device 2600 described above may be supplemented with a wireless communication module 3210, such as a short-range communications subsystem, and a digital signal processor (DSP) or other communication interface component as part of the controller 2690. The short-range communication subsystem may use a Zigbee protocol, a Bluetooth protocol, a personal area network protocol, a Wi-Fi protocol, an IEEE 802.11-compliant protocol or another suitable low-power, short range wireless communications protocol.

The controller 2690 in such embodiments may comprise suitable hardware and execute suitable software to process control commands received via the wireless communication module 3210. If a valid command is determined by the controller 2690 to have been received via the wireless communication module 3210, then the controller 2690 operates the pump 2687 or the pressure relief valve 2688 (as appropriate) in response to the valid command. If no valid command is determined to have been received, no action is taken by the controller 2690. The commands may be received at the wireless communication module 3210 from an external control device, such as a handheld computing device 3220 (for example, a smart phone or a laptop or tablet computer). Such commands may be received at the wireless communication module 3210 following exchange of handshaking signals 3215 between the wireless communication module 3210 and the handheld computing device 3220. The handheld computing device 3220 may execute an application (i.e. an “App”) specifically designed to interface with the clamping device 3200 and for this purpose, the controller 2690 may be configured to send data back to the handheld computing device 3220 via the wireless communication module 3210. Such data may include sensed pressure data, device status data or other device operational data, such as power level of the batteries, component malfunctions detected (if any), number of uses of the device 3200 since a last reset (to determine when it should be cleaned next), etc. Although not shown, some embodiments may include a sensor to indicate to the controller 2690 when the device 2600/3200 has been placed in a clamped position or an unclamped position.

In some embodiments, the clamping device 2600 may include a visual and/or audio indicator and a processor. The visual indicator is capable of producing a visible output for humans and the audio indicator is capable of producing an audible output for humans. In some embodiments, the indicator includes a light emitting device (LED) and/or a speaker. The indicator is connected to the pressure sensor 2110 and the processor and device 2600 may also include a user input to allow a user to set a predetermined pressure value. The indicator may be triggered by the processor to indicate when the sensed pressure reaches the predetermined pressure. Triggering the indicator may include lighting up the LED and/or producing a human audible sound. The provision of an indicator assists an operator of the clamping device 2600 in reliably applying a clamping pressure to the arm at the predetermined pressure value. An insufficient clamping pressure may lead to inaccurate blood pressure measurements taken with clamping device 2600 and sphygmomanometer 2410.

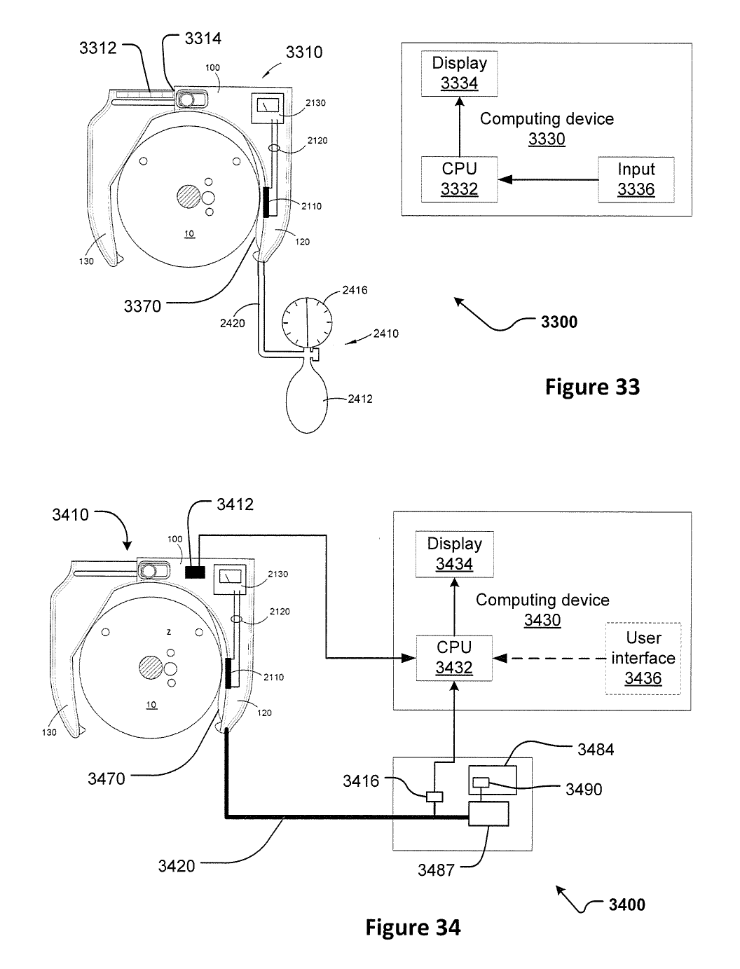

Referring to FIG. 33, there is provided a blood pressure monitoring system 3300 comprising device 3310, sphygmomanometer 2410 and computing device 3330 according to some embodiments. Device 3310 may be the same as device 2100 except that it includes expandable element 3370 and at least one sensing component. Expandable element 3370 is connectable to the sphygmomanometer 2410 and may be identical to expandable element 2470.

As described earlier, the pressure sensor 2110 can be used to reliably apply a predetermined clamping pressure on the human upper arm 10. In some embodiments, if the expandable element 2470 is partially inflated (E.g. to a pressure up to 20 mmHg), the pressure sensor 3416 that is pneumatically coupled to the expandable element can be used to determine the clamping pressure. The pressure sensor 3416 is electrically coupled with CPU 3432 which can be used to trigger an indicator when the clamping pressure has reached the predetermined pressure as described earlier.

Pressure values indicated on display 2416 on the sphygmomanometer 2410 may be used to determine a set of uncorrected blood pressure values (i.e. the systolic and diastolic blood pressure values) in human upper arm 10. For example, the clamping device 3310 can be used to apply a clamping pressure to the arm 10, via the inflatable element 3370, that is above the systolic blood pressure of the subject being tested. The clamping pressure can then be removed and uncorrected blood pressure values may be determined with the sphygmomanometer 2410 using either the auscultatory or oscillometric method and reading pressure values from the pressure indicator 2416.

The at least one sensing component in device 3410 may include a spatial sensing component adapted to sense or indicate a spatial dimension value of the limb being clamped such as the diameter or circumference. In some embodiments, the spatial sensing component is an indicator component which may comprise a mechanical meter with an elongate pointer such as a needle for indicating the spatial dimension value on a graduated scale or dial. For example, the indicator component may be a dial meter or panel meter. The mechanical meter may be coupled to both jaws 120 and 130 such that the needle moves when the jaws 120 and 130 move relative to each other to indicate the spatial dimension value on the scale or dial. In some embodiments, the indicator may include a graduated scale 3312 on the external surface of jaw 130 that indicates the spatial dimension value by the location at which the extreme end 3314 of the jaw 120 overlaps the graduated scale 3312. As further detailed below, the spatial dimension value, when the clamping device 3310 has been clamped on the arm 10 at the pre-determined pressure, can be used in calculations of corrected blood-pressure values.

In some embodiments, the at least one sensing component includes a spatial sensor that is capable of producing an output indicative of the spatial dimension value of the arm 10. The output can be provided to display 2130 to display the spatial dimension value. The display 2130 may, for example, include an electro-mechanical or digital display.

The computing device 3330 includes an input component 3336, a processor such as a central processing unit (CPU) 3332, and a display 3334. The input component 3336 is connected to the processor and comprises a user interface configured to receive input from a user of the computing device 3330. In some embodiments, the user interface includes a touch screen, buttons or a keypad. A pressure value indicative of the sensed pressure indicated on the display 2130 may be sent via input component 3336 to the CPU 3332. The set of uncorrected blood pressure values obtained from pressure indicator 2416 may be sent via input component 3336 to the CPU 3332. A user of device 3310 can also read the spatial dimension value from the graduated scale 3312 or display 2130 and send the values to the CPU 3332 by inputting the values using input component 3336. The computing device 3330 uses the input values to calculate corrected blood pressure values which are then displayed on a display 3334 on the computing device 3330.

In some embodiments, the computing device 3330 may be a portable hand-held device. Computing device 3330 may be a purpose built device or a generic computer, calculator, tablet computer or mobile telephone (i.e. a ‘smart phone’) that includes a programmable storage medium for storing instructions to implement the functions described above.

In some embodiments, any other suitable means may be used to calculate the corrected blood pressure values. For example, a user may use the spatial dimension and pressure values indicated on displays 2130 and 2416 in non-computer aided calculations or referring to a look-up table to determine the corrected blood pressure values.

In an alternative embodiment illustrated in FIG. 34, a blood pressure monitoring system 3400 includes a clamping device 3410, a computing device 3430 and an electronic sphygmomanometer 3440. Clamping device 3410 may be the same as clamping device 3310 except that a spatial sensor 3412 is coupled to the processor 3432 of the computing device 3430 so that the spatial dimension value indicative of the dimension of the arm 10 can be sent to the CPU 3432 to calculate a set of corrected blood pressure values to be displayed on display 3434 as described above. For example, sensor output from sensor 3412 indicative of the spatial dimension value, is sent to the computing device 3430 and the computing device 3430 uses the spatial dimension value to calculate corrected blood pressure values along with the set of blood pressure values obtained from the electronic sphygmomanometer 3440 as described in further detail below. In some embodiments, clamping device 3410 does not include a display 2130.

The computing device 3430 may be the same as computing device 3330 except that the CPU 3432 is coupled to the clamping device 3410 and the electronic sphygmomanometer 3440. The computing device may optionally comprise a user interface 3436 for manual input of pressure values. This may be useful if, for example, the electronic sphygmomanometer 3440 is non-functional and a manual sphygmomanometer 2410 is used to obtain blood pressure measurements.

The electronic sphygmomanometer 3440 is configured to inflate/deflate the expandable element 3470 via fluid conduit 3420 to effect blood pressure measurements in an autonomous or semiautonomous manner. The electronic sphygmomanometer 3440 comprises control functions to automate the inflation and deflation of the expandable element 3470 similar to those described for clamping device 2600. Such control functions may be provided by a controller 3490 on a circuit board 3484 that is electrically coupled to a pump 3487 to operate the pump 3487 to pump air into (to inflate) the expandable element 3470. The controller 3490 can automate a further inflation of the expandable element 3470 to a pressure above the systolic pressure in the arm 10 and then can automate the progressive deflation of the expandable element 3470. In some embodiments, the circuit board 3484 is part of a blood pressure monitoring module.

The electronic sphygmomanometer 3440 further comprises a pressure sensor 3416 coupled to fluid conduit 3420. The pressure sensor 3416 is adapted produce and send a output indicative of the sensed pressure, during progressive deflation of the expandable element 3470, to CPU 3432 for calculation of a set of blood pressure values using the oscillometric method. The data indicative of the blood pressure values along with the spatial dimension pressure value from the sensor 3412 is used by the CPU 3432 to calculate a corrected blood pressure value in arm 10 that may then be displayed on display 3434.

In some embodiments, the computing device 3430 may be the same as computing device 3330 except that the CPU 3432 is only coupled to the electronic sphygmomanometer 3440. In these embodiments, the clamping device 3410 does not include a spatial sensor 3412 and instead a meter or graduated scale is provided as described above. The spatial dimension value is then read from the meter or scale and input into the CPU 3432 via user interface 3436.

In some embodiments, the electronic sphygmomanometer 3440 may include a processor to calculate a set of uncorrected blood pressure values and the set of uncorrected values may be sent to the CPU 3432. The electronic sphygmomanometer 3440 may also comprise a display for displaying the set of uncorrected blood pressure values.

In some embodiments, the pump 3487 includes a pressure sensing function. The pump 3487 provides an output signal to computing device 3430 indicative of the sensed pressure in the expandable element 3470. The CPU 3432 of the computing device 3430 uses the output signal from pump 3487 to calculate the blood pressure values of the subject as described earlier.

In some embodiments, the computing device 3430 may be co-located with the electronic sphygmomanometer 3440 and form at least part of a blood pressure monitoring module. In some embodiments, either one of or both of the computing device 3430 and electronic sphygmomanometer 3440 may be co-located with or part of the clamping device 3410. In some embodiments, any one or more components of the computing device 3430 may be part of the circuit board 3484. The circuit board 3484 may be part of a blood pressure monitoring module. In some embodiments, the pressure sensor 3416 may also be part of the circuit board 3484.

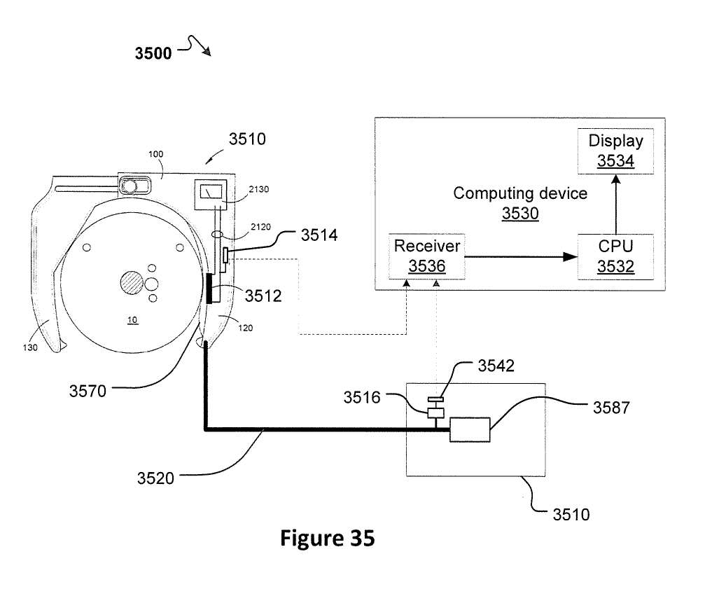

Referring to FIG. 35, a system 3500 according to another embodiment is shown. System 3500 comprises clamping device 3510, computing device 3530 and electronic sphygmomanometer 3540.

The clamping device 3510 is similar to device 3410 but includes a first transmitter 3514 that is coupled to sensor 3512. The first transmitter 3514 is configured to transmit data corresponding to the clamping pressure value sensed from the sensor 3512 to computing device 3530. The computing device 3530 includes a receiver 3536 configured to receive the data corresponding to the pressure value from the device 3510. The electronic sphygmomanometer 3540 is coupled to the expandable element 3570 via fluid conduit 3520 and is similar to electronic sphygmomanometer 3440 but includes a second transmitter 3542. The second transmitter 3542 is coupled to pressure sensor 3516 that senses the pressure within the expandable element 3570 and is configured to transmit data indicative of the pressure values obtained by sensor 3516 to the computing device 3520. The data may be transmitted via any suitable short-range (low power) wireless protocol such as Bluetooth or through a wireless network.

In some embodiments, the electronic sphygmomanometer 3540 is located within the clamping device 3510 (and therefore part of the components of the clamping device) and the sensor 3512 is electrically conductively coupled to the electronic sphygmomanometer 3540. The electronic sphygmomanometer 3540 uses output from the sensor 3512 to determine the set of blood pressure values from the arm 10 of the human subject and an output indicative of any one of the set of blood pressure values, spatial dimension values and clamping pressure values may be transmitted to the computing device 3530 to calculate a set of corrected blood pressure values.

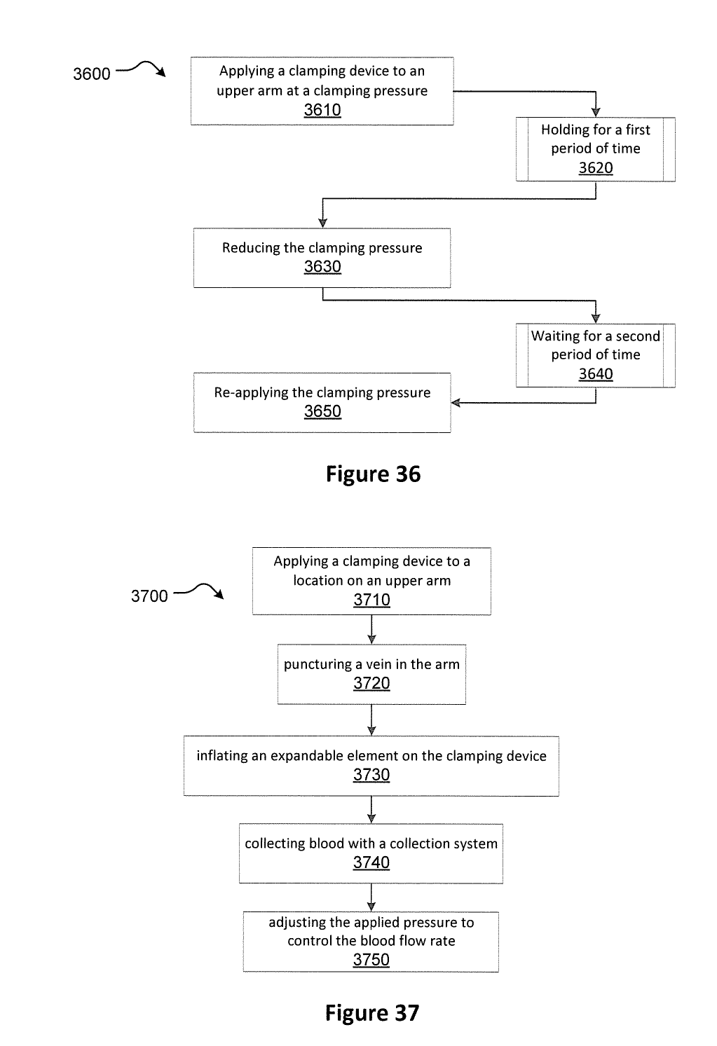

Referring to FIG. 36, a method of improved venous dilation 3600 is provided. At 3610, the method comprises applying a clamping device around an upper section of an arm (upper arm 10) of a human subject at a first clamping pressure to create some level of hypoxia in the arm. For example, some level of hypoxia is created in the section of the arm distal to the location where the clamping device is placed on the arm. The upper section of the arm may, for example, be the section of the arm between the shoulder and the elbow. The clamping pressure is applied at a pressure approximately equal to the systolic arterial pressure of the arteries in the upper arm 10. At 3620, this condition is held for a first period of time of more than 20 seconds. The method further comprises releasing the first clamping pressure or reducing the first clamping pressure of the clamping device at 3630 by a predetermined percentage of the first clamping pressure within a predetermined time to allow arterial oxygenised blood to flow into the upper arm 10, and waiting for a second period of more than 5 seconds at 3640. Subsequent to the second period, the clamping device is again applied at a second clamping pressure less than the first clamping pressure to improve venous dilation in the arm. For example, venous dilation is improved in the section of the arm distal to the location where the clamping device is placed on the arm. Advantageously, the veins in the arm dilate not only due to venous constriction but also due to hypoxia. This may aid in venepuncture for blood sample collection or blood donations by making it easier to locate and puncture veins in the arm.

In some embodiments, the first clamping pressure is set to an expected systolic blood pressure value of the human subject. Humans with a normal blood pressure range have a systolic blood pressure between 90 and 120 mmHg. However, humans the systolic blood pressure may be as low as 60 mmHg and as high as 180 mmHg. The first clamping pressure may therefore be set to a value between 60 and 180 mmHg. In some embodiments, the method 3600 further includes determining the systolic pressure value in the arm 10 using the clamping device herein described or any other means and the first clamping pressure is between 80% and 120% of the systolic pressure value determined. In some embodiments, the first clamping pressure is between 90% and 110% of the systolic pressure value determined and optionally 100%.

Preferably, the first clamping pressure is substantially released at 3630. In some embodiments, the step 3630 comprises reducing the first clamping pressure in the order of 30-80%. In some embodiments, the first clamping pressure is reduced by at least 80%. In other embodiments, the first clamping pressure is reduced by 90-100%. In some embodiments the first clamping pressure is reduced in less than 3 seconds or less than 1 second.

In some embodiments the first period is between 20-60 seconds. In some embodiments the second period is between 5-60 seconds. In some embodiments the second period is 10-30 seconds and optionally 15 seconds.

In some embodiments, the second clamping pressure is greater than or equal to the diastolic blood pressure and less than the systolic pressure in the in the upper arm 10. In some embodiments the second clamping pressure is set according to the expected diastolic blood pressure in a human subject. Typically, the diastolic blood pressure in humans is between 60 and 140 mmHg and the second clamping pressure is therefore set between 60 and 140 mmHg. In some embodiments, the second clamping pressure is set according to: the expected diastolic blood pressure of the human subject with low blood pressure (between 60 and 80 mmHg); normal blood pressure (between 80 and 110 mmHg); or high blood pressure (110 and 140 mmHg).

As an example, if the second clamping pressure is set to 120 mmHg and the human subject has a set of high blood pressure values of 180/110 (systolic blood pressure/diastolic blood pressure), then the second clamping pressure is about 67% of the systolic blood pressure. In another example, if the second clamping pressure is set to 81 mmHg and the human subject has a set of low blood pressure values of 90/60 (systolic blood pressure/diastolic blood pressure), then the second clamping pressure is about 90% of the systolic blood pressure.

Typically, the diastolic blood pressure value in humans is 66% of the systolic blood pressure value. In some embodiments, the method comprises determining the blood pressure values in the upper arm 10 using the clamping device herein described or any other means and the second clamping pressure is between 60% and 90% of the systolic pressure value determined. In some embodiments, the second clamping pressure is between 70% and 90% of the systolic pressure value determined. In some embodiments the second clamping pressure is between 80% and 90% of the systolic pressure value determined.

In some embodiments, to implement the method of improved venous dilation the clamping device may be any one of clamping devices 2600, 3310, 3410 or 3510. In some embodiments, the controller 2690 or 3490 may be programed to inflate or deflate the expandable element 2470, 2670, 3370, 3470 or 3570 according to the earlier described method.

In some embodiments, the method 3600 may comprise applying an automated inflatable cuff in place of the clamping device. The automated inflatable cuff includes an elongate strap comprising an inflatable bladder which is connected to a pump. The pump is controlled by a controller. The strap is capable of extending around at least the entire circumference of an upper arm of a human and includes a component for securing the strap around the upper arm. When applied to the upper arm, inflation of the inflatable bladder by the pump results in the application of pressure to the upper arm, this pressure is the equivalent of the clamping pressure applied by the clamping device discussed above. The automated inflatable cuff operates in the same way as described for the clamping device when used in method 3600.

Referring to FIG. 37, a method 3700 of controlling the rate of venous blood outflow is described. The method 3700 comprises applying a clamping device 2600, 3310, 3410 or 3510 or an automated inflatable cuff at a location on an upper section of the arm (upper arm 10) as earlier described, at 3710; and puncturing a vein in the arm with a needle at a point distal from the location on the upper arm 10 that the clamping device is applied, at 3720. The location of where the vein is punctured is in the section of the arm distal to the location where the clamping device or the automated inflatable cuff is placed on the arm. With the needle in position, at step 3730, expandable element 2470, 2670, 3370, 3470, or 3570 is inflated to apply an applied pressure that is below the arterial pressure. In some embodiments, the applied pressure may be 50 mmHg. As the applied pressure is below the arterial pressure, blood continues to flow distally past the location of the clamping device into the limb which increases venous pressure. When venous pressure increases to the applied pressure, blood will flow into the needle. The method 3700 also includes collecting blood with a collection system at 3740, and adjusting the applied pressure to control the blood flow rate at 3750.

In some embodiments, the blood collection system is capable of measuring the blood flow rate and data indicative of the blood flow rate may be provided by a flow meter in the blood collection system to the clamping device 2600, 3310, 3410 or 3510. The controller 2690 or 3490 may be programmed to adjust the applied pressure based on the data indicative of the blood flow rate. For example, the controller 2690 or 3490 may be programmed to maintain a pre-determined blood flow rate. In other examples, the controller 2690 or 3490 may be programmed to adjust the blood flow rate according to a pre-determined timing schedule.

Some device embodiments may be suitably described as a non-encircling tourniquet, which may be implemented with two opposing movable plates. The plates may be connected via a telescoping, lockable bridge section, which allows for relative movement in a direction largely perpendicular to a common plane of the plates. The plates, bridge and locking mechanism thus effectively combine to form a clamping device. The plates are preferably shaped in such a fashion they provide specific structure to assist in compressing the veins of the (upper) arms. In this regard, the structure of the plates may be configured to provide pressure points that correlate to the location of the major veins in the arms. Such structures are strategically placed on the inside or arm side of the clamping device to achieve venous compression and at least partial venous stasis. These pressure points result in needing less pressure provided by the operator of the device, prior to venepuncture.

Embodiments may also employ a sliding sleeve or insert originating from the medial inner plate that extends along the inside of the plates and bridge, comprising a 3-4 mm raised central longitudinal spine aiding in venous compression. The sleeve or insert may protrude through the distal end of the lateral plate. Upon closing or tightening the device into a clamped position, the sleeve will protrude further through the distal lateral plate and the amount of protrusion is largely proportional to the tightening movement. This protrusion and movement is in reaction to the decreasing inner circumference of the clamping device at the bridge and aides in avoiding or minimising pinching, grabbing or injuring the skin of the arm on to which the clamping device is applied. A side button release mechanism may be provided for easy detachment.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A clamping device for applying pressure to a human limb comprising:
a first rigid part having a first inner profile,
a second rigid part generally facing the first inner profile, wherein the first and second inner profiles are arranged to apply clamping pressure against the human limb when the device is in the clamped position and thereby apply clamping pressure to blood vessels in the limb;
a coupling portion that couples the first and second rigid parts together while allowing relative movement of the first and second parts between a clamped position and an unclamped position;
an expandable element arranged at least partly along at least one of the first inner profile and the second inner profile; wherein the expandable element is inflatable to apply additional pressure to the limb, and deflatable to reduce the additional pressure, when the clamping device is positioned on the limb; and
at least one sensing component.

2. The clamping device of claim 1, wherein the at least one sensing component includes a spatial sensing component adapted to sense or indicate a spatial dimension value for either a diameter or circumference of the human limb.

3. The clamping device according to claim 2, wherein the clamping device includes a meter for indicating the spatial dimension value.

4. (canceled)

5. The clamping device of claim 2, wherein the spatial sensing component is a spatial sensor adapted to provide a first output indicative of a spatial dimension value for the spatial dimension.

6. The clamping device according to claim 1, wherein the clamping device further comprises a pressure sensing component comprising a pressure sensor for sensing pressure.

7. The clamping device of claim 6, wherein the pressure sensor is adapted to provide a second output indicative of pressure.

8. The clamping device of claim 7, wherein the second output comprises an output indicative of the clamping pressure applied by the clamping device to the human limb.

9. The clamping device according to claim 8, wherein the clamping device further comprises:
a processor adapted to receive the second output; and
an indicator,
wherein the processor is configured to trigger the indicator to indicate when a predetermined clamping pressure is reached.

10. The clamping device according to claim 9, wherein the indicator provides at least one of a visible or audible output.

11. The clamping device of claim 5, wherein the clamping device is adapted to provide at least one of the first output or second output to a computing device.

12. The clamping device of claim 11, wherein at least one of the spatial sensor and the pressure sensor are adapted to be electrically coupled to the computing device to thereby provide the at least one of the first and second output.

13. The clamping device of claim 11, wherein the clamping device further comprises at least one transmitter for providing the at least one of the first and second output to the computing device.

14. The clamping device according to claim 5, wherein the clamping device further comprises a display coupled to the at least one sensing component to receive at least one of the first output and second output to thereby display at least one of the spatial dimension value and the clamping pressure value.

15. The clamping device of claim 14, wherein the display includes either an electro-mechanical meter or an electronic display.

16. The clamping device according to claim 1, wherein the expandable element is adapted to be connected to a sphygmomanometer for inflating and deflating the expandable element and to determine a set of blood pressure values.

17. The clamping device according to claim 1, wherein the clamping device further comprises:
a pump in fluid connection with the expandable element for inflating and deflating the expandable element; and
a controller connected to the pump for controlling the pump to inflate and deflate the expandable element in a predetermined manner for blood pressure measurements.

18. The clamping device according to claim 17, wherein the at least one sensing component includes a pressure sensor and a spatial sensing component adapted to sense or indicate a spatial dimension value for either a diameter or circumference of the human limb,
wherein the clamping device further comprises:
a processor electrically coupled to the pressure sensor,
wherein the pressure sensor is pneumatically coupled to the expandable element, and
wherein the controller and the processor are adapted to determine a set of corrected blood pressure values using the second output from the pressure sensor and the spatial dimension value.

19. The clamping device according to claim 8, wherein the at least one sensing component includes a spatial sensing component adapted to sense or indicate a spatial dimension value for either a diameter or circumference of the human limb,
wherein the clamping device further comprises:
a pump in fluid connection with the expandable element for inflating and deflating the expandable element;
a controller connected to the pump for controlling the pump to inflate and deflate the expandable element in a predetermined manner for blood pressure measurements;
a further pressure sensor pneumatically coupled to the expandable element; and
a processor electrically coupled to the further pressure sensor,
wherein the controller and the processor are adapted to determine a set of corrected blood pressure values using an output from the further pressure sensor and the spatial dimension value.

20. The clamping device of claim 18, wherein the spatial sensing component is a spatial sensor coupled to the processor and adapted to provide a first output indicative of a spatial dimension value of the spatial dimension to the processor.

21. (canceled)

22. A blood pressure monitoring system for calculating a corrected blood pressure value in a human limb, the system comprising:
the clamping device according to claim 1, and
a computing device for calculating the corrected blood pressure value, wherein the computing device includes:
an input component configured to receive a first value indicative of a cross-sectional dimension of the human limb, a second value indicative of a clamping pressure applied by the clamping device, and a set of blood pressure values,
a first processor; and
a storage medium including instructions for the processor to calculate a set of corrected blood pressure values using the first value, second value and set of blood pressure values.

23-34. (canceled)
